(19) United States
(12) Patent Application Publication
(10) Pub. No.: US 2011/0087000 A1
(43) Pub. Date: Apr. 14, 2011

(54) Integrated Process to Selectively Convert Renewable Isobutanol to P-Xylene

(75) Inventors: Matthew W. Peters, Highlands Ranch, CO (US); Joshua D. Taylor, Evergreen, CO (US); Madeline Jenni, Parker, CO (US); Leo E. Manzer, Lewes, DE (US); David E. Henton, Midland, MI (US)

(73) Assignee: GEVO, Inc., Englewood, CO (US)

(21) Appl. No.: 12/899,285

(22) Filed: Oct. 6, 2010

Related U.S. Application Data
(60) Provisional application No. 61/249,078, filed on Oct. 6, 2009.
(60) Provisional application No. 61/295,886, filed on Jan. 18, 2010.
(60) Provisional application No. 61/352,228, filed on Jun. 7, 2010.

(51) Int. Cl. C08G 63/127; C07C 1/24; C07C 51/255

(52) U.S. Cl. 528/308.3; 585/240; 562/412

(57) ABSTRACT

The present invention is directed to a method for preparing renewable and relatively high purity p-xylene from biomass. For example, biomass treated to provide a fermentation feedstock is fermented with a microorganism capable of producing a C₄ alcohol such as isobutanol, then sequentially dehydrating the isobutanol in the presence of a dehydration catalyst to provide a C₄ alkene such as isobutylene, dimerizing the C₄ alkene to a form one or more C₈ alkenes such as 2,4,4-trimethylpentenes or 2,5-dimethylhexene, then dehydrocyclizing the C₈ alkenes in the presence of a dehydrocyclization catalyst to selectively form renewable p-xylene in high overall yield. The p-xylene can then be oxidized to form terephthalic acid or terephthalate esters.

### CROSS REFERENCE TO RELATED APPLICATIONS

The present application claims priority to U.S. Provisional Application Nos. 61/249,078 filed Oct. 6, 2009, 61/295,886 filed Jan. 18, 2010, and 61/352,228 filed Jun. 7, 2010, the disclosures of each of which are herein incorporated by reference in their entireties for all purposes.

### BACKGROUND OF THE INVENTION

Aromatic compounds are conventionally produced from petroleum feedstocks in refineries by reacting mixtures of light hydrocarbons (C₁-C₆) and naphthas over various catalysts at high heat and pressure. The mixture of light hydrocarbons available to a refinery is diverse, and provides a mixture of aromatic compounds (e.g., BTEX—benzene, toluene, ethylbenzene, and xylenes, as well as aromatic compounds having a molecular weight higher than xylenes). The xylenes product consists of three different aromatic C₈ isomers: p-xylene, o-xylene, and m-xylene; typically about one third of the xylenes are the p-xylene isomer. The BTEX mixture is then subjected to subsequent processes to obtain the desired product. For example, toluene can be removed and disproportionated to form benzene and xylene, or the individual xylene isomers can be isolated by fractionation (e.g. by absorptive separation, fractional crystallization, etc.). p-Xylene is the most commercially important xylene isomer, and is used almost exclusively in the production of polyester fibers, resins, and films. o-Xylene and m-xylene are also used in the production of phthalic anhydride, and isophthalic acid, respectively.

Alternatively, a single component feedstock purified from crude oil or synthetically prepared at the refinery can be selectively converted to purer aromatic product. For example, pure isooctene can be selectively aromatized to form primarily p-xylene over some catalysts (see, for example, U.S. Pat. No. 3,202,725, U.S. Pat. No. 4,229,320, U.S. Pat. No. 4,247,726, U.S. Pat. No. 6,600,081, and U.S. Pat. No. 7,067,708), and n-octane purified from crude oil can be converted to primarily o-xylene (see for example, U.S. Pat. No. 2,785,209).

Very high p-xylene purity is required to prepare terephthalic acid of suitable purity for use in polyester production—typically at least about 95% pure, or in some cases 99.7% or higher purity of p-xylene is required. Conventional processes for producing high purity p-xylene are thus complex and expensive: the conventional BTEX process requires isolation and extensive purification of p-xylene produced at relatively low levels; and alternative processes require isolation and purification of single component feedstocks for aromatization from complex hydrocarbon mixtures. Furthermore, production of p-xylene from conventional petroleum-based feedstocks contributes to environmental degradation (e.g., global warming, air and water pollution, etc.), and fosters over-dependence on unreliable petroleum supplies from politically unstable parts of the world. The present invention provides a simple process for preparing renewable, high purity p-xylene from renewable carbon sources, which can be converted to terephthalic acid and polyesters.

### SUMMARY OF THE INVENTION

In one embodiment, the present invention is directed to a process for preparing renewable p-xylene comprising:
(a) treating biomass to form a fermentation feedstock;
(b) fermenting the fermentation feedstock with one or more species of microorganism to form a fermentation broth comprising aqueous isobutanol;
(c) removing aqueous isobutanol from the fermentation broth;
(d) dehydrating, in the presence of a dehydration catalyst, at least a portion of the aqueous isobutanol of step (c), thereby forming a dehydration product comprising one or more C₄ alkenes and water;
(e) dimerizing, in the presence of an oligomerization catalyst, a dimerization feedstock comprising at least a portion of the C₄ alkenes formed in step (d), thereby forming a dimerization product comprising one or more C₈ alkenes (optionally containing unreacted C₄ alkenes, and optionally comprising 2,4,4-trimethylpentenes, 2,5-dimethylhexene(s), and/or 2,5-dimethylhexadiene(s);
(f) dehydrocyclizing, in the presence of a dehydrocyclization catalyst, a dehydrocyclization feedstock comprising at least a portion of the C₈ alkenes of step (e), thereby forming a dehydrocyclization product comprising xylenes and hydrogen (and optionally one or more unreacted C₄ alkenes, unreacted 2,4,4-trimethylpentene(s), 2,5-dimethlyhexene(s), and/or 2,5-dimethylhexadiene(s)), wherein the xylenes comprise at least about 75% p-xylene.

In another embodiment, the present invention is also directed to methods for preparing renewable terephthalic acid from renewable p-xylene prepared by the method of the present invention.

In still another embodiment, the present invention is directed to methods for preparing renewable polyester terephthalate from the renewable terephthalic acid prepared by the method of the present invention.

### BRIEF DESCRIPTION OF THE DRAWINGS

FIG. 1 is a schematic diagram of one embodiment of a process of the present invention for preparing p-xylene from isobutanol.

FIG. 2 is a schematic diagram of a single pass process according to the present invention for preparing p-xylene from isobutanol.

FIG. 3 is a schematic diagram of a single pass process according to the present invention for preparing p-xylene from isobutanol, which includes yields for various intermediates and products in the process.

FIG. 4 is a schematic diagram of an integrated process according to the present invention, as described in Example 15.

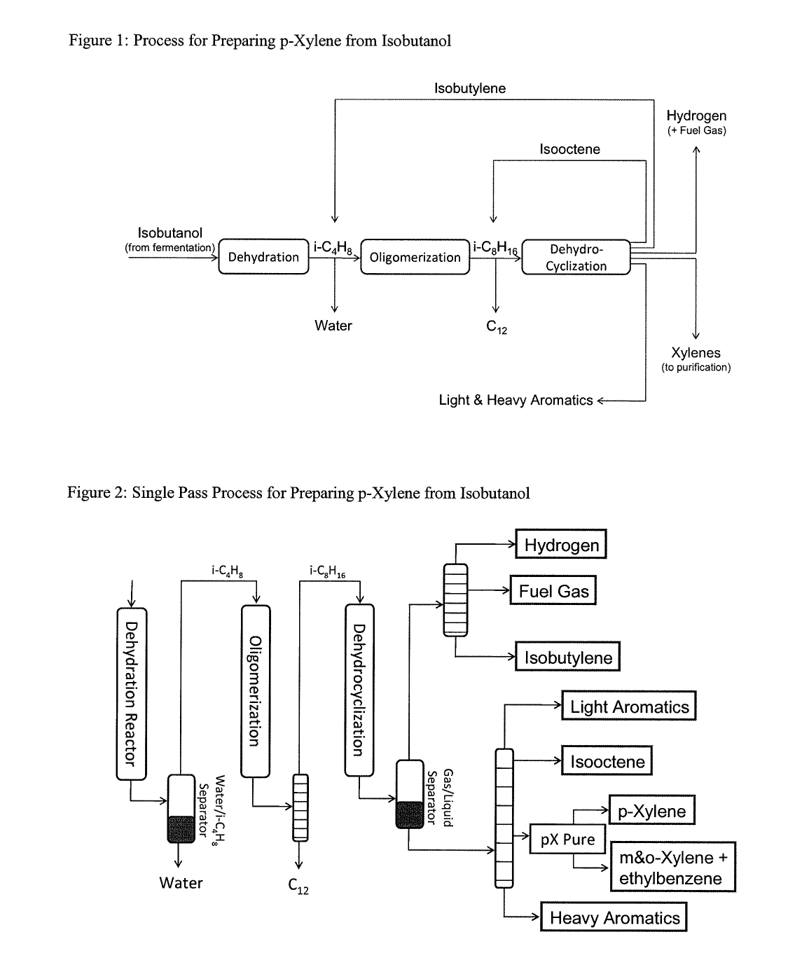

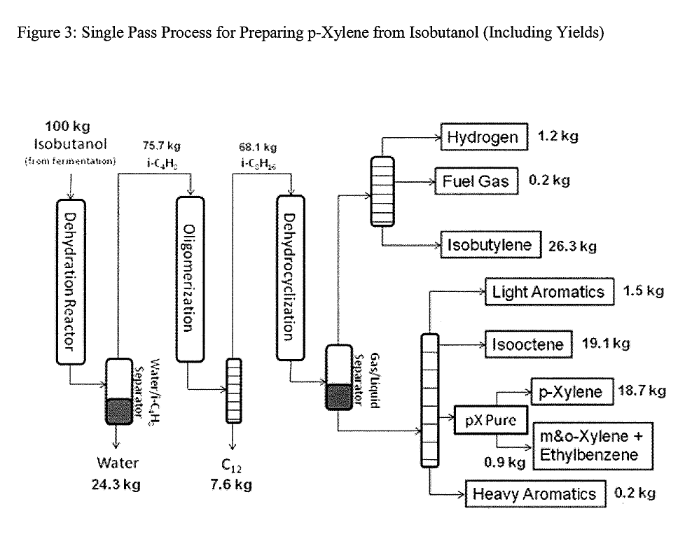

### DETAILED DESCRIPTION OF THE INVENTION

All documents disclosed herein (including patents, journal references, ASTM methods, etc.) are each incorporated by reference in their entirety for all purposes.

The term “biocatalyst” means a living system or cell of any type that speeds up chemical reactions by lowering the activation energy of the reaction and is neither consumed nor altered in the process. Biocatalysts may include, but are not limited to, microorganisms such as yeasts, fungi, bacteria, and archaea.

The biocatalyst herein disclosed can convert various carbon sources into precursors for p-xylene. The term “carbon source” generally refers to a substance suitable for use as a source of carbon for prokaryotic or eukaryotic cell growth. Carbon sources include, but are not limited to biomass hydrolysates, starch, sucrose, cellulose, hemicellulose, xylose, and lignin, as well as monomeric components of these substrates (e.g., monosaccharides). Carbon sources can comprise various organic compounds in various forms including, but not limited to, polymers, carbohydrates, acids, alcohols, aldehydes, ketones, amino acids, peptides, etc. These include, for example, various monosaccharides such as glucose, dextrose (D-glucose), maltose, oligosaccharides, polysaccharides, saturated or unsaturated fatty acids, succinate, lactate, acetate, ethanol, etc., or mixtures thereof. Photosynthetic organisms can additionally produce a carbon source as a product of photosynthesis. In some embodiments, carbon sources may be selected from biomass hydrolysates and glucose.

The term “feedstock” is defined as a raw material or mixture of raw materials supplied to process for subsequent conversion into an intermediate or a final product. For example, a carbon source, such as biomass or the carbon compounds derived from biomass (e.g., a biomass hydrolysate as described herein) is a feedstock for a biocatalyst (e.g., a microorganism) in a fermentation process, and the resulting alcohol (e.g., isobutanol) produced by the fermentation can be a feedstock for subsequent unit operations (e.g., dehydration as described herein): e.g., isobutylene resulting from the dehydration of isobutanol can be a feedstock for dimerization, and the resulting diisobutylene (e.g., 2,4,4-trimethylpentene(s), 2,5-dimethylhexene(s), 2,5-dimethylhexadiene(s), etc.) can be a feedstock for dehydrocyclization. A feedstock may comprise one or more components. For example, the feedstock for a fermentation process (i.e., a fermentation feedstock) typically contains nutrients other than the carbon source; the feedstock for a dehydration unit operation typically also comprises water, the feedstock for dehydration typically also comprises water, the feedstock for dimerization typically also comprises diluents and unreacted isobutanol, and the feedstock for dehydrocyclization also typically comprises diluents, unreacted isobutanol and isobutylene, etc. The term “fermentation feedstock” is used interchangeably with the term “renewable feedstock”, as fermentation feedstocks are generated from biomass or traditional carbohydrates, which are renewable substances.

The term “traditional carbohydrates” refers to sugars and starches generated from specialized plants, such as sugar cane, corn, and wheat. Frequently, these specialized plants concentrate sugars and starches in portions of the plant, such as grains, that are harvested and processed to extract the sugars and starches. Traditional carbohydrates such as those derived from corn are co-produced with food products derived from the protein-rich portion of the grains, and are primarily used as renewable feedstocks for fermentation processes to generate biofuels or fine chemicals (or precursors thereof).

Alternatively, renewable alcohols can be prepared photosynthetically, e.g., using cyanobacteria or algae engineered to produce isobutanol, isopentanol, and/or other alcohols (e.g., Synechococcus elongatus PCC7942 and Synechocystis PCC6803; see Angermayr et al., Energy Biotechnology with Cyanobacteria, Current Opinion in Biotechnology 2009, 20, 257-263, Atsumi and Liao, Nature Biotechnology, 2009, 27, 1177-1182); and Dexter et al., Energy Environ. Sci., 2009, 2, 857-864, and references cited in each of these references). When produced photosynthetically, the “feedstock” for producing the resulting renewable alcohols is light and the CO₂ provided to the photosynthetic organism (e.g., cyanobacteria or algae).

The term “biomass” as used herein refers primarily to the stems, leaves, and starch-containing portions of green plants, and is mainly comprised of starch, lignin, cellulose, hemicellulose, and/or pectin. Biomass can be decomposed by either chemical or enzymatic treatment to the monomeric sugars and phenols of which it is composed (Wyman, C. E. 2003 Biotechnological Progress 19:254-62). This resulting material, called biomass hydrolysate, is neutralized and treated to remove trace amounts of organic material that may adversely affect the biocatalyst, and is then used as a feedstock for fermentations using a biocatalyst. Alternatively, the biomass may be thermochemically treated to produce alcohols, alkanes, and alkenes that may be further treated to produce p-xylene.

The term “starch” as used herein refers to a polymer of glucose readily hydrolyzed by digestive enzymes. Starch is usually concentrated in specialized portions of plants, such as potatoes, corn kernels, rice grains, wheat grains, and sugar cane stems.

The term “lignin” as used herein refers to a polymer material, mainly composed of linked phenolic monomeric compounds, such as p-coumaryl alcohol, coniferyl alcohol, and sinapyl alcohol, which forms the basis of structural rigidity in plants and is frequently referred to as the woody portion of plants. Lignin is also considered to be the non-carbohydrate portion of the cell wall of plants.

The term “cellulose” as used herein refers is a long-chain polymer polysaccharide carbohydrate comprised of β-glucose monomer units, of formula (C₆H₁₀O₅)ₙ, usually found in plant cell walls in combination with lignin and any hemicellulose.

The term “hemicellulose” refers to a class of plant cell-wall polysaccharides that can be any of several heteropolymers. These include xylane, xyloglucan, arabinoxylan, arabinogalactan, glucuronoxylan, glucomannan and galactomannan. Monomeric components of hemicellulose include, but are not limited to: D-galactose, L-galactose, D-mannose, L-rhamnose, L-fucose, D-xylose, L-arabinose, and D-glucuronic acid. This class of polysaccharides is found in almost all cell walls along with cellulose. The molecular weight of hemicellulose is lower than for cellulose. Hemicellulose cannot be extracted with hot water or chelating agents, but can be extracted by aqueous alkali. Polymeric chains of hemicellulose bind pectin and cellulose in a network of cross-linked fibers forming the cell walls of most plant cells.

The term “pectin” as used herein refers to a class of plant cell-wall heterogeneous polysaccharides that can be extracted by treatment with acids and chelating agents. Typically, 70-80% of pectin is found as a linear chain of α-(1-4)-linked D-galacturonic acid monomers. The smaller RG-I fraction of pectin is comprised of alternating (1-4)-linked galacturonic acid and (1-2)-linked L-rhamnose, with substantial arabinogalactan branching emanating from the rhamnose residue. Other monosaccharides, such as D-fucose, D-xylose, apiose, aceric acid, Kdo, Dha, 2-O-methyl-D-fucose, and 2-O-methyl-D-xylose, are found either in the RG-II pectin fraction (<2%), or as minor constituents in the RG-I fraction. Proportions of each of the monosaccharides in relation to D-galacturonic acid vary depending on the individual plant and its micro-environment, the species, and time during the growth cycle. For the same reasons, the homogalacturonan and RG-I fractions can differ widely in their content of methyl esters on GalA residues, and the content of acetyl residue esters on the C-2 and C-3 positions of GalA and neutral sugars.

The term “yield” is defined as the amount of product obtained per unit weight of raw material and may be expressed as g product/g substrate. Yield may also be expressed as a percentage of the theoretical yield. “Theoretical yield” is defined as the maximum amount of product that can be generated per a given amount of substrate as dictated by the stoichiometry of the metabolic pathway used to make the product. For example, if the theoretical yield for one typical conversion of glucose to isobutanol is 0.41 g/g, the yield of isobutanol from glucose of 0.39 g/g would be expressed as 95% of theoretical or 95% theoretical yield.

The terms “alkene” and “olefin” are used interchangeably herein to refer to non-aromatic hydrocarbons having at least one carbon-carbon double bond.

“Renewably-based” or “renewable” denote that the carbon content of the indicated compound is from a “new carbon” source as measured by ASTM test method D 6866-08, “Standard Test Methods for Determining the Bio-Based Content of Solid, Liquid, and Gaseous Samples Using Radiocarbon Analysis”. This test method measures the ¹⁴C/¹²C isotope ratio in a sample and compares it to the ¹⁴C/¹²C isotope ratio in a standard 100% biobased material to give percent biobased content of the sample. A small amount of the carbon atoms of the carbon dioxide in the atmosphere is the radioactive isotope ¹⁴C. This ¹⁴C carbon dioxide is created when atmospheric nitrogen is struck by a cosmic ray generated neutron, causing the nitrogen to lose a proton and form carbon of atomic mass 14 (¹⁴C), which is then immediately oxidized to carbon dioxide. A small but measurable fraction of atmospheric carbon is present in the form of ¹⁴CO₂. Atmospheric carbon dioxide is processed by green plants to make organic molecules during the process known as photosynthesis. Virtually all forms of life on Earth depend on this green plant production of organic molecule to produce the chemical energy that facilitates growth and reproduction. Therefore, the ¹⁴C that forms in the atmosphere eventually becomes part of all life forms and their biological products, enriching biomass and organisms which feed on biomass with ¹⁴C. In contrast, carbon from “fossil” petroleum-based hydrocarbons does not have the signature ¹⁴C:¹²C ratio of renewable organic molecules derived from atmospheric carbon dioxide, because ¹⁴C eventually decays to ¹⁴N (t_{1/2} of 5730 years).

“Biobased materials” are organic materials in which the carbon comes from recently (on a human time scale) fixated CO₂ present in the atmosphere using sunlight energy (photosynthesis). For example, a biobased hydrocarbon has a ¹⁴C/¹²C isotope ratio greater than 0. Contrarily, a fossil-based hydrocarbon has a ¹⁴C/¹²C isotope ratio of about 0. The term “renewable” with regard to compounds such as alcohols or hydrocarbons (e.g., alkenes, aromatics, etc.) refers to compounds prepared from biomass using thermochemical methods (e.g., gasification of biomass to form “syngas”, which is subsequently reacted with Fischer-Tropsch catalysts to form e.g., hydrocarbons, alcohols, etc.), biocatalysts (e.g., fermentation), or other processes, for example as described herein.

The application of ASTM-D6866-08 to derive “biobased content” is built on the same concepts as radiocarbon dating, but without use of the age equations. The analysis is performed by deriving a ratio of the amount of radiocarbon (¹⁴C) in an unknown sample compared to that of a modern reference standard. This ratio is reported as a percentage with the units “pMC” (percent modern carbon). If the material being analyzed is a mixture of present day radiocarbon and fossil carbon (containing very low levels of radiocarbon), then the pMC value obtained correlates directly to the amount of biomass material present in the sample.

The p-xylene prepared by the methods of the present invention has pMC values of at least about 1, 5, 10, 15, 20, 25, 30, 35, 40, 45, 50, 55, 60, 65, 70, 75, 80, 85, 90, 95, 100, inclusive of all values and subranges therebetween. In one embodiment, the pMC value of the p-xylene prepared by the methods of the present invention is greater than about 90; in another embodiment, the pMC value of the p-xylene prepared by the methods of the present invention is greater than about 95; in yet another embodiment, the pMC value of the p-xylene prepared by the methods of the present invention is greater than about 98; in still yet another embodiment, the pMC value of the p-xylene prepared by the methods of the present invention is greater than about 99; in a particular embodiment, the pMC value of the p-xylene prepared by the methods of the present invention is about 100.

The term “dehydration” refers to a chemical reaction that converts an alcohol into its corresponding alkene. For example, the dehydration of isobutanol produces isobutylene.

The term “dimerization” or “dimerizing” refer to oligomerization processes in which two identical activated molecules (such as isobutylene) are combined with the assistance of a catalyst (a dimerization catalyst or oligomerization catalyst, as described herein) to form a larger molecule having twice the molecular weight of either of the starting molecules (such as diisobutylene or 2,4,4-trimethylpentenes). The term “oligomerization” can be used to refer to a “dimerization” reaction, unless the formation of oligomers other than dimers is expressly or implicitly indicated.

The term “aromatization” refers to processes in which hydrocarbon starting materials, typically alkenes or alkanes are converted into one or more aromatic compounds (e.g., p-xylene) in the presence of a suitable catalyst by dehydrocyclization.

“Dehydrocyclization” refers to a reaction in which an alkane or alkene is converted into an aromatic hydrocarbon and hydrogen, usually in the presence of a suitable dehydrocyclization catalyst, for example any of those described herein.

The term “reaction zone” refers to the part of a reactor or series of reactors where the substrates and chemical intermediates contact a catalyst to ultimately form product. The reaction zone for a simple reaction may be a single vessel containing a single catalyst. For a reaction requiring two different catalysts, the reaction zone can be a single vessel containing a mixture of the two catalysts, a single vessel such as a tube reactor which contains the two catalysts in two separate layers, or two vessels with a separate catalyst in each which may be the same or different.

The phrase “substantially pure p-xylene” refers to isomeric composition of the xylenes produced by the dehydrocyclization step of the process. Xylenes which comprise “substantially pure p-xylene” comprise at least about 75% of the p-xylene isomer; and accordingly less than about 25% of the xylenes are other xylene isomers (e.g., o-xylene and m-xylene). Thus, xylenes comprising “substantially pure p-xylene” can comprise about 75%, about 80%, about 85%, about 90%, about 95%, about 96%, about 97%, about 98%, about 99%, about 99.5%, about 99.9%, or about 100% p-xylene.

The term “conversion” refers to the degree to which the reactants in a particular reaction (e.g., dehydration, dimerization, dehydrocyclization, etc.) are converted to products. Thus 100% conversion refers to complete consumption of reactants, and 0% conversion refers to no reaction.

The term “selectivity” refers to the degree to which a particular reaction forms a specific product, rather than another product. For example, for the dehydration of isobutanol, 50% selectivity for isobutylene means that 50% of the alkene products formed are isobutylene, and 100% selectivity for isobutylene means that 100% of the alkene products formed are isobutylene. Because the selectivity is based on the product formed, selectivity is independent of the conversion or yield of the particular reaction.

“WHSV” refers to weight hourly space velocity, and equals the mass flow (units of mass/hr) divided by catalyst mass. For example, in a dehydration reactor with a 100 g dehydration catalyst bed, an isobutanol flow rate of 500 g/hr would provide a WHSV of 5 hr⁻¹.

Unless otherwise indicated, all percentages herein are by weight (i.e., “wt. %).

In most embodiments, the fermentation feedstock comprises a carbon source obtained from treating biomass. Suitable carbon sources include any of those described herein such as starch, mono- and polysaccharides, pre-treated cellulose and hemicellulose, lignin, and pectin etc., which are obtained by subjecting biomass to one or more processes known in the art, including extraction, acid hydrolysis, enzymatic treatment, etc.

The carbon source is converted into a precursor of p-xylene (such as isobutanol) by the metabolic action of the biocatalyst (or by thermochemical methods, e.g. using gasification followed by chemical reaction over Fischer-Tropsch catalysts). The carbon source is consumed by the biocatalyst (e.g., a microorganism as described herein) and excreted as a p-xylene precursor (e.g., isobutanol) in a large fermentation vessel. The p-xylene precursor is then separated from the fermentation broth, optionally purified, and then subjected to further processes such as dehydration, dimerization, and aromatization to form aromatics comprising substantially pure p-xylene.

Depending on the biocatalyst, a particular C₄ alcohol or a mixture of C₄ alcohols can be obtained. For example, the biocatalyst can be a single microorganism capable of forming more than one type of C₄ alcohol during fermentation (e.g. two or more of 1-butanol, isobutanol, 2-butanol, t-butanol, etc.). In most embodiments however, it is most advantageous to obtain primarily one type of C₄ alcohol. In a particular embodiment, the C₄ alcohol is isobutanol. Accordingly, in most embodiments, a particular microorganism which preferentially forms isobutanol during fermentation is used.

Alternatively, renewable butanols (e.g., isobutanol) are prepared photosynthetically using an appropriate photosynthetic organism (cyanobacteria or algae as described herein).

Any suitable organism which produces a C₄ alcohol can be used in the fermentation step of the process of the present invention. For example, alcohols such as isobutanol are produced by yeasts during the fermentation of sugars into ethanol. Such alcohols (termed fusel alcohols in the art of industrial fermentations for the production of beer and wine) have been studied extensively for their effect on the taste and stability of these products. Recently, production of fusel alcohols using engineered microorganisms has been reported (U.S. Patent Publication No. 2007/0092957, and Nature, 2008, 451, p. 86-89). Isobutanol can be fermentatively produced by recombinant microorganisms as described in U.S. Provisional Patent Application No. 60/730,290 or in U.S. Patent Publ. Nos. 2009/0226990, 2009/0226991, 2009/0215137, 2009/0171129; 2-butanol can be fermentatively produced by recombinant microorganisms as described in U.S. Patent Application No. 60/796,816; and 1-butanol can be fermentatively produced by recombinant microorganisms as described in U.S. Provisional Patent Application No. 60/721,677. Other suitable microorganisms include those described, for example in U.S. Patent Publ. Nos. 2008/0293125, 2009/0155869.

The C₄ alcohol produced during fermentation can be removed from the fermentation broth by various methods, for example fractional distillation, solvent extraction (e.g., in particular embodiments with a renewable solvent such as renewable oligomerized hydrocarbons, renewable hydrogenated hydrocarbons, renewable aromatic hydrocarbons, etc. prepared as described herein), adsorption, pervaporation, etc. or by combinations of such methods, prior to dehydration. In other embodiments, the alcohol produced during fermentation is not isolated from the fermentation broth prior to dehydration, but is dehydrated directly as a dilute aqueous solution.

In a particular embodiment, the C₄ alcohol is removed by the process described in U.S. Patent Publ. No. 2009/0171129 A1. Specifically, the C₄ alcohol can be removed from the fermentation broth by either increasing the thermodynamic activity of the C₄ alcohol and/or decreasing the thermodynamic activity of the water, for example, maintaining the headspace of the fermentation vessel, or a side-stream of fermentation broth removed from the fermentation vessel (e.g., using a flash tank or other apparatus), at reduced pressure (e.g., below atmospheric pressure), and/or heating the side-stream of the fermentation broth, thereby providing a vapor phase comprising water and the C₄ alcohol (e.g., aqueous isobutanol). In a particular embodiment, the vapor phase provided thereby consists essentially of water and the C₄ alcohol. In yet another particular embodiment, the vapor phase provides an azeotropic mixture of the water and the C₄ alcohol. The vapor phase comprising the C₄ alcohol and water can be fed directly to the dehydration reaction step, or can be further concentrated by, for example cooling to condense the water and the C₄ alcohol to produce a two-phase liquid composition comprising a C₄ alcohol-rich phase, and a water-rich phase. The C₄ alcohol-rich liquid phase can then be separated from the water-rich phase using various methods known in the art, e.g., a liquid-liquid separator, etc. The aqueous C₄ alcohol removed from the fermentor can be further purified to remove water and/or other contaminants from the fermentation process, using conventional methods such as distillation, absorption, pervaporation, etc.

The removal of C₄ alcohol from the fermentation broth, as described herein, can occur continuously or semi-continuously. Removal of the C₄ alcohol in the manner described herein is advantageous because it provides for separation of the C₄ alcohol from the fermentation broth without the use of relatively energy intensive or equipment intensive unit operations such as distillation, pervaporation, absorption, etc., and removes a metabolic by-product of the fermentation, thereby improving the productivity of the fermentation process.

After removing the C₄ alcohol(s) from the fermentor, the C₄ alcohol(s) are converted to p-xylene by first catalytically dehydrating the alcohol to C₄ alkene(s) (isobutylene, 1-butene, and/or 2-butene), then catalytically dimerizing the C₄ alkene(s) to C₈ alkene(s) (linear or branched octenes, 2,4,4-trimethylpentenes, 2,5-dimethylhexenes, 2,5-dimethylhexadienes, etc.). The C₈ alkene(s) are finally reacted in the presence of a dehydrocyclization catalyst to selectively form p-xylene. As is described in more detail herein, in particular embodiments the dehydration, dimerization, and dehydrocyclization reaction steps are carried out under reaction conditions which favor selectively forming specific products. For example, the dehydration reaction is carried out in the presence of a particular dehydration catalyst (as described herein), and under particular temperature, pressure, and WHSV conditions which selectively form isobutylene (e.g., at least about 95% of the C₄ alkenes formed are isobutylene); the dimerization reaction is carried out in the presence of a particular dimerization catalyst (as described herein), and under particular temperature, pressure, diluent and WHSV conditions which selectively form 2,4,4-trimethylpentenes, 2,5-dimethylhexenes, and/or 2,5-dimethylhexadienes (e.g., at least about 50% of the C₈ alkenes formed are 2,4,4-trimethylpentenes, 2,5-dimethylhexenes, and/or 2,5-dimethylhexadienes); and the dehydrocyclization reaction is carried out in the presence of a particular dimerization catalyst (as described herein), and under particular temperature, pressure, diluent and WHSV conditions which selectively form p-xylene (e.g., at least about 75% of the xylenes formed are p-xylene).

Selective dehydration, dimerization, and dehydrocyclization reaction steps are promoted by a variety of methods which reduce unwanted side-reactions (and the resulting undesirable by-products), such as the use of particularly selective catalysts, the addition of diluents, reduced reaction temperatures, reduced reactant residence time over the catalyst (i.e., higher WHSV values), etc. Such reaction conditions tend to reduce the percent conversion of particular reaction steps below 100%, and thus the feedstock for each successive reaction can include unreacted starting materials from the previous reaction step (which can function as diluents, as well as added diluents and by-products from previous reaction steps; For example, the feedstock for the dehydrocyclization reaction step can include the C₈ alkene produced by a dimerization reaction, as well as diluent gases (e.g., nitrogen, argon, and methane), unreacted C₄ alkene, etc. from the dimerization reaction, by-product C₄ and/or C₈ alkane from the dehydrocyclization reaction, etc. Unreacted starting materials can also be recycled back to the appropriate reaction step in order to boost the overall yield of p-xylene. For example, unreacted C₄ alkene present in the product stream from the dimerization reaction (or in some cases, also present in the product stream from the dehydrocyclization reaction) can be separated out of the product stream and recycled back to the feedstock for the dimerization reaction. In addition, C₄ and C₈ alkane by-products formed during the dehydrocyclization reaction (e.g., from the corresponding C₄ and C₈ alkenes present in the dehydrocyclization feedstock) can be recycled back to the feedstock for the dehydrocyclization reaction. C₈ alkanes (e.g., isooctane, 2,5-dimethylhexenes, 2,5-dimethylhexadienes, etc.) can react in the presence of the dehydrocyclization catalyst to form p-xylene, and C₄ alkene functions as a relatively inert diluent. The C₄ alkane can be recycled back to the feedstock of the oligomerization reaction where it acts as a diluent, which increases the selectivity of the oligomerization reaction, thereby providing products which are selectively dehydrocyclized to p-xylene.

The various reaction steps subsequent to production of the C₄ alcohol (e.g., dehydration, dimerization, and dehydrocyclization) can be carried out in a single reactor, within which the individual reaction steps take place in different reaction zones; or in which the catalysts are mixed or layered together in a single reaction zone, whereby the C₄ alcohol undergoes sequential conversion to successive intermediates in a single reaction zone (e.g., conversion of the C₄ alcohol to a C₄ alkene, then a C₈ alkene in a single reaction zone; or conversion of a C₄ alkene to a C₈ alkene, then dehydrocyclization of the C₈ alkene to p-xylene in a single reaction zone). Alternatively, the various reactions can be carried out in separate reactors so that the reactor conditions (e.g., temperature, pressure, catalyst, feedstock composition, WHSV, etc.) can be optimized to maximize the selectivity of each reaction step. When the separate reaction steps are carried out in separate reactors, the intermediates formed in the various reaction steps can be isolated and/or purified before proceeding to the subsequent reaction step, or the reaction product from one reactor can be passed directly to the subsequent reactor without purification.

In other embodiments of the processes of the present invention, one or more of the particular reaction steps (e.g., dehydration, dimerization, dehydrocyclization) can each be carried out in two or more reactors (connected either in series or in parallel), so that during operation of the process, particular reactors can be bypassed (or taken “offline”) to allow maintenance (e.g., catalyst regeneration) to be carried out on the bypassed reactor, while still permitting the process to continue in the remaining operational reactors. For example, the dehydrocyclization step could be carried out in two reactors connected in series (whereby the product of the dimerization step is the feedstock for the first dehydrocyclization reactor, and the product of the first dehydrocyclization reactor is the feedstock for the second dehydrocyclization reactor). The first dehydrocyclization reactor can be bypassed using the appropriate piping and valves such that the product of the dehydrocyclization step is now the feedstock for the second dehydrocyclization reactor. For reactors connected in parallel, bypassing one of the reactors may simply entail closing the feed and product lines of the desired reactor. Such reactor configurations, and means for by-passing or isolating one or more reactors connected in series or parallel are known in the art.

The C₄ alcohol feedstock for the dehydration reaction can comprise a single C₄ alcohol (e.g., isobutanol) or can comprise a mixture of C₄ alcohols. In most embodiments, the dehydration feedstock comprises a single C₄ alcohol (e.g., isobutanol).

The dehydration reaction catalytically converts the C₄ alcohol produced in the fermentation step (e.g. isobutanol) into the corresponding C₄ alkene (e.g., isobutylene). Depending upon the dehydration catalyst used, dehydration of the C₄ alcohol can also be accompanied by rearrangement of the resulting C₄ alkene to form one or more isomeric alkenes. If isomerization occurs, the isomerization can occur concurrently with the dehydration, or subsequently to the dehydration.

The dehydration of alcohols to alkenes can be catalyzed by many different catalysts. In general, acidic heterogeneous or homogeneous catalysts are used in a reactor maintained under conditions suitable for dehydrating the C₄ alcohol. Typically, the C₄ alcohol is activated by an acidic catalyst to facilitate the loss of water. The water is usually removed from the dehydration reactor with the product. The resulting C₄ alkene either exits the reactor (e.g., in the gas or liquid phase depending upon the reactor conditions) and is captured by a downstream purification process or is further converted in the reactor to other compounds as described herein. For example, t-butyl alcohol is dehydrated to isobutylene by reacting it in the gas phase at 300-400° C. over an acid treated aluminum oxide catalyst (U.S. Pat. No. 5,625,109) or in the liquid phase at 120-200° C. over a sulfonic acid cationic exchange resin catalyst (U.S. Pat. No. 4,602,119). The water generated by the dehydration reaction exits the reactor with unreacted C₄ alcohol and C₄ alkene product and is separated by distillation or phase separation. Because water is generated in large quantities in the dehydration step, the catalysts used are generally tolerant to water and a process for removing the water from substrate and product may be part of any process that contains a dehydration step. For this reason, it is possible to use wet (i.e., up to 99% water by weight) C₄ alcohol as a substrate for a dehydration reaction and remove this water with the water generated by the dehydration reaction. For example, dilute aqueous solutions of ethanol (up to 98% water by weight) can be dehydrated over a zeolite catalyst with all water removed from the ethylene product stream after the dehydration step occurs (U.S. Pat. Nos. 4,698,452 and 4,873,392). Additionally, neutral alumina and zeolites will dehydrate alcohols to alkenes. For example, neutral chromium treated alumina will dehydrate isobutanol to isobutylene above 250° C. (U.S. Pat. No. 3,836,603).

Levels of water between about 0% and about 15% have little if any effect on the percent conversion and selectivity of the subsequent dehydration reaction. In most embodiments, the feedstock for the dehydration reaction comprises an aqueous C₄ alcohol comprising about 0-15% water, including about 0% water, about 1% water, about 2% water, about 3% water, about 4% water, about 5% water, about 6% water, about 7% water, about 8% water, about 9% water, about 10% water, about 11% water, about 12% water, about 13% water, about 14% water, or about 15% water, inclusive of all ranges and subranges therebetween. In a particular embodiment, the aqueous C₄ alcohol feedstock for the dehydration reaction comprises aqueous isobutanol containing about 0-15% water. In a specific embodiment, the dehydration reaction feedstock consists essentially of aqueous isobutanol containing about 0-15% water (e.g., about 85-100% isobutanol, and about 0-15% water), and trace levels of impurities (for example less than about 5% impurities, e.g., less than about 4%, less than about 3%, less than about 2%, or less than about 1% impurities).

Suitable dehydration catalysts include homogeneous or heterogeneous catalysts. A non-limiting list of homogeneous acid catalysts include inorganic acids such as sulfuric acid, hydrogen fluoride, fluorosulfonic acid, phosphotungstic acid, phosphomolybdic acid, phosphoric acid, Lewis acids such as aluminum and boron halides (e.g., AlCl₃, BF₃, etc.); organic sulfonic acids such as trifluoromethanesulfonic acid, p-toluenesulfonic acid and benzenesulfonic acid; heteropolyacids; fluoroalkyl sulfonic acids, metal sulfonates, metal trifluoroacetates, compounds thereof and combinations thereof. A non-limiting list of heterogeneous acid catalysts include heterogeneous heteropolyacids (HPAs); solid phosphoric acid; natural clay minerals, such as those containing alumina or silica; cation exchange resins such as sulfonated polystyrene ion exchange resins; metal oxides, such as hydrous zirconium oxide, Fe₂O₃, Mn₂O₃, γ-alumina, etc.; mixed metal oxides, such as sulfated zirconia/γ-alumina, alumina/magnesium oxide, etc.; metal salts such as metal sulfides, metal sulfates, metal sulfonates, metal nitrates, metal phosphates, metal phosphonates, metal molybdates, metal tungstates, metal borates; zeolites, such as NaY zeolite, H-ZSM-5, NaA zeolite, etc.; modified versions of any of the above known in the art, and combinations of any of the above, for example as described in U.S. Publ. Nos. 2009/0030239, 2008/0132741, 2008/0132732, 2008/0132730, 2008/0045754, 2008/0015395.

The dehydration reaction of the processes of the present invention is typically carried out using one or more fixed-bed reactors using any of the dehydration catalysts described herein. Alternatively, other types of reactors known in the art can be used, such as fluidized bed reactors, batch reactors, catalytic distillation reactors, etc. In a particular embodiment, the dehydration catalyst is a heterogeneous acidic γ-alumina catalyst. In order to maximize the purity of p-xylene ultimately produced, and to reduce or eliminate the need for purification of intermediates, it is desirable to carry out the dehydration reaction under conditions which favor selective formation of isobutylene. Higher selectivity is favored at lower conversion and under milder dehydration conditions (e.g., lower temperature and pressure).

In some embodiments, the dehydration reaction is carried out in the vapor phase to facilitate removal of water (either present in the dehydration feedstock or as a by-product of the dehydration reaction). In most embodiments, the dehydration reaction is carried out at a pressure ranging from 0-30 psig, and at a temperature of about 350° C. or less (e.g., about 300-350° C.). In other embodiments, the dehydration reaction pressure is about 0, about 5, about 10, about 15, about 20, about 25, or about 30, inclusive of all ranges and subranges therebetween. In most embodiments, the dehydration reaction temperature is about 325° C. or less, about 300° C. or less, about 275° C. or less, or about 250° C. or less. In a specific embodiment, the dehydration temperature is about 300° C. In another particular embodiment, the dehydration temperature is about 275° C. In still other embodiments, the dehydration temperature is at least about 100° C. and a pressure of at least about 1 atm.

The weight hourly space velocity (WHSV) of the dehydration reaction can range from about 1 to about 10 hr⁻¹, or about 1, about 2, about 3, about 4, about 5, about 6, about 7, about 8, about 9, or about 10 hr⁻¹. In a specific embodiment, the WHSV is about 5 hr⁻¹.

In still other embodiments, the dehydration reaction is carried out at higher pressures, ranging from about 60 psig to about 200 psig, for example at about 60 psig, about 70 psig, about 80 psig, about 90 psig, about 100 psig, about 110 psig, about 120 psig, about 130 psig, about 140 psig, about 150 psig, about 160 psig, about 170 psig, about 180 psig, about 190 psig, or about 200 psig, inclusive of all ranges and subranges therebetween. When the dehydration reaction is carried out at such pressures, the isobutylene and water of the dehydration reaction product are separated in a liquid-liquid separator.

If the dehydration reaction product, or portions of the dehydration reaction product are produced in the vapor phase, the C₄ alkene (e.g. isobutylene) and water components of the dehydration reaction product can be separated by gas-liquid or liquid-liquid separation methods (i.e. after condensing the dehydration reaction product by cooling and/or compression). If the dehydration reaction product is substantially liquid, the product forms a C₄ alkene (e.g. isobutylene) rich phase and a water rich phase, which can be separated using a liquid-liquid separator.

In order for the processes of the present invention to ultimately provide substantially pure p-xylene, it is desirable to carry out the dehydration reaction under “selective” process conditions (e.g., choice of catalyst(s), temperature, pressure, WHSV, etc.) which provide a C₄ alkene product which is primarily isobutylene. In particular embodiments, the combination of temperature, pressure, catalyst used, and WHSV are selected such that the C₄ alkene product comprises at least about 95% isobutylene, e.g., temperatures of about 300° C. or lower, pressures of about 0-80 psig, catalysts such as BASF AL-3996, and a WHSV of about 5 hr⁻¹. In other particular embodiments, the C₄ alkene product comprises at least about 96%, at least about 97%, at least about 98%, at least about 99%, or about 100% isobutylene, inclusive of all ranges and sub-ranges therebetween.

The water produced in the dehydration reaction can be separated from the C₄ alkene (e.g., isobutylene) by various methods. For example, if the dehydration reaction is carried out at pressures of about 0-30 psig, the C₄ alkene can be separated as a gas from liquid water using a gas-liquid separator. When the dehydration reaction is carried out at pressures of about 30-100 psig, both the C₄ alkene and water can be condensed (e.g., by cooling or compressing the product stream) and the separation carried out using a liquid-liquid separator. In particular embodiments, the C₄ alkene (e.g., isobutylene) and water are separated after dehydration by gas-liquid separation. In some embodiments, unreacted C₄ alcohol is recycled back to the dehydration feedstock after separation from the C₄ alkene.

In particular embodiments, the dehydration reaction is run at temperature/pressure conditions (e.g., temperatures of about 250-350° C., pressures of 60-200 psig, WHSV of about 1-20 hr⁻¹). The C₄ alkene (e.g., isobutylene) product is then separated from the aqueous phase using a liquid-liquid separator. At least a portion of the unreacted isobutanol can be recycled back to the dehydration reaction feed; a portion of the unreacted isobutanol remaining in the C₄ alkene product mixture can also be retained in the dehydration product stream, and act as a diluent and/or modifier in the dimerization feedstock to improve selectivity of the dimerization reaction step.

In another particular embodiment, the dehydration reaction is carried out in multiple separate reactors (e.g., two, three, or more dehydration reactors) connected in series, wherein the temperature of the reactors increases in each successive dehydration reactor. When configured in this manner, one or more of the dehydration reactors can be bypassed during operation to permit e.g., regeneration of a “coked” catalyst in the bypassed reactor, without requiring a shutdown of the overall process.

In other embodiments, instead of recycling the unreacted isobutanol from the dehydration product stream, at least a portion of the unreacted isobutanol obtained after separation from the C₄ alkene (e.g., by liquid-liquid or gas-liquid separation) can be further dehydrated in additional dehydration reactors, and the resulting C₄ alkene product added to the feedstock for the dimerization step.

In most embodiments the dehydration and dimerization steps are carried out separately. In other embodiments, the dehydration and dimerization reactions are carried out in a single reaction zone using a catalyst (or mixture of catalysts) which catalyzes both reactions. The C₄ alkene(s) formed in the dehydration step can be transferred directly to the oligomerization catalyst (e.g., in another reaction zone or another reactor), or can be isolated prior to dimerization. In one embodiment, the C₄ alkene is isolated as a liquid and optionally purified (e.g., by distillation) prior to dimerization. Isolation of the C₄ alkene can be advantageous if the dehydration process is optimally carried out under gas-phase conditions, whereas the dimerization is optimally carried out under liquid-phase conditions; thus isolation of the C₄ alkene allows the dehydration and dimerization reactions to each be carried out under optimal conditions. Isolation of the C₄ alkene can refer to a process in which the C₄ alcohol produced by the biocatalyst (or thermochemical process) is continuously removed from the fermentor (as described herein) and dehydrated continuously to provide C₄ alkene. The C₄ alkene can then be stored and later reacted further (e.g., oligomerization and/or aromatization and/or hydrogenation and/or oxidation), or the isolated C₄ alkene can be temporarily stored in a holding tank prior to e.g. oligomerization providing an integrated, continuous process in which each of the unit operations (e.g., fermentation, dehydration, oligomerization, dehydrocyclization, etc.) run simultaneously and more or less continuously, and the isolation of the C₄ alkene “buffers” process upsets.

The oligomerization catalyst catalyzes dimerization, trimerization, etc. of the C₄ alkene. In the process of the present invention, primarily dimerization of the C₄ alkene to C₈ alkene(s) (e.g., 2,4,4-trimethylpentenes, etc.) is favored by appropriate selection of oligomerization catalyst and process conditions. In most embodiments, the dimerization reaction step is carried out under conditions which favor substantially exclusive dimer product (i.e., at least about 90% of the oligomers formed are C₈ alkene, at least about 95% of the oligomers formed are C₈ alkene, at least about 98% of the oligomers formed are C₈ alkene, at least about 99% of the oligomers are C₈ alkene, or about 100% of the oligomers formed are C₈ alkene). The unreacted C₄ alkene is then recycled.

Furthermore, the dimerization process is carried under selective conditions in which the C₈ alkene formed comprises primarily 2,4,4-trimethylpentenes; that is, the C₈ alkene dimers comprise at least about 50% 2,4,4-trimethylpentenes, or at least about 55%, at least about 60%, at least about 65%, at least about 70%, at least about 75%, at least about 80%, at least about 85%, at least about 90%, at least about 95%, or about 100% 2,4,4-trimethylpentenes.

In other embodiments, the dimerization process is carried under selective conditions in which the C₈ alkene formed comprises primarily 2,5-dimethylhexenes; that is, the C₈ alkene dimers comprise at least about 50% 2,5-dimethylhexenes, or at least about 55%, at least about 60%, at least about 65%, at least about 70%, at least about 75%, at least about 80%, at least about 85%, at least about 90%, at least about 95%, or about 100% 2,5-dimethylhexenes.

In still other embodiments, the dimerization process is carried under selective conditions in which the C₈ alkene formed comprises primarily 2,5-dimethylhexadienes; that is, the C₈ alkene dimers comprise at least about 50% 2,5-dimethylhexadienes, or at least about 55%, at least about 60%, at least about 65%, at least about 70%, at least about 75%, at least about 80%, at least about 85%, at least about 90%, at least about 95%, or about 100% 2,5-dimethylhexadienes.

In further embodiments, the dimerization process is carried under selective conditions in which the C₈ alkene formed comprises primarily 2,5-dimethylhexenes and 2,5-dimethylhexadienes; that is, the C₈ alkene dimers comprise at least about 50% 2,5-dimethylhexenes and 2,5-dimethylhexadienes, or at least about 55%, at least about 60%, at least about 65%, at least about 70%, at least about 75%, at least about 80%, at least about 85%, at least about 90%, at least about 95%, or about 100% 2,5-dimethylhexenes and 2,5-dimethylhexadienes.

At the high conversion conditions typical in petrochemical processing (e.g., >95% conversion), the oligomerization product typically comprises a mixture of isooctenes and isododecenes, which would require isolation and purification of the isooctene component prior to dehydrocyclization in order to provide sufficiently pure p-xylene. The selective dimerization conditions as described herein provide high levels of diisobutylene, for example 2,4,4,-trimethylpentenes, 2,5-dimethylhexenes, or 2,5-dimethylhexadienes, which can be converted subsequently to substantially pure p-xylene by dehydrocyclization as described herein. Selective dimerization conditions which produce essentially exclusively dimer alkene product, comprising at least about 50% 2,4,4-trimethylpentenes, 2,5-dimethylhexenes, or 2,5-dimethylhexadienes (or in other embodiments, at least about 55%, at least about 60%, at least about 65%, at least about 70%, at least about 75%, at least about 80%, at least about 85%, at least about 95%, at least about 95%, or about 100% 2,4,4-trimethylpentenes, 2,5-dimethylhexenes, or 2,5-dimethylhexadienes, inclusive of all ranges and subranges therebetween) are provided by various means, for example catalyst selection, choice of temperature and/or pressure, WHSV, the presence of diluents and modifiers, and combinations thereof. Suitable selective dimerization conditions include, for example dimerization with an Amberlyst strongly acidic ionic exchange resin catalyst at a temperature of about 100-120° C., approximately atmospheric pressure, WHSV of about 10-50 hr⁻¹, and a feedstock comprising about 50-90% diluents; for a ZSM-5 catalyst (e.g. CBV 2314), suitable dimerization conditions include a reaction temperature of about 150-180° C., a pressure of about 750 psig, a WHSV of about 10-100 hr⁻¹, and a feedstock comprising about 30-90% diluents; and for a solid phosphoric acid catalyst, suitable conditions include a reaction temperature of about 160-190° C., a pressure of about 500-1000 psig, WHSV of about 10-100 hr⁻¹, and a feedstock comprising about 25-75% diluents.

A non-limiting list of suitable acidic oligomerization catalysts includes inorganic acids, organic sulfonic acids, heteropolyacids, perfluoroalkyl sulfonic acids, metal salts thereof, mixtures of metal salts, and combinations thereof. The acid catalyst may also be selected from the group consisting of zeolites such as CBV-3020, ZSM-5, β Zeolite CP 814C, ZSM-5 CBV 8014, ZSM-5 CBV 5524 G, and YCBV 870; fluorinated alumina; acid-treated silica; acid-treated silica-alumina; acid-treated titania; acid-treated zirconia; heteropolyacids supported on zirconia, titania, alumina, silica; and combinations thereof. The acid catalyst may also be selected from the group consisting of metal sulfonates, metal sulfates, metal trifluoroacetates, metal triflates, and mixtures thereof; mixtures of salts with their conjugate acids, zinc tetrafluoroborate, and combinations thereof.

Other acid catalysts that may be employed in dimerization step of the invention include inorganic acids such as sulfuric acid, phosphoric acid (e.g., solid phosphoric acid), hydrochloric acid, and nitric acid, as well as mixtures thereof. Organic acids such as p-toluene sulfonic acid, triflic acid, trifluoroacetic acid and methanesulfonic acid may also be used. Moreover, ion exchange resins in the acid form may also be employed. Hence, any type of suitable acid catalyst known in the art may be employed.

Fluorinated sulfonic acid polymers can also be used as acidic oligomerization catalysts for the dimerization step of the processes of the present invention. These acids are partially or totally fluorinated hydrocarbon polymers containing pendant sulfonic acid groups, which may be partially or totally converted to the salt form. One suitable fluorinated sulfonic acid polymer is Nafion® perfluorinated sulfonic acid polymer, (E.I. du Pont de Nemours and Company, Wilmington, Del.). Another suitable fluorinated sulfonic acid polymer is Nafion® Super Acid Catalyst, a bead-form strongly acidic resin which is a copolymer of tetrafluoroethylene and perfluoro-3,6-dioxa-4-methyl-7-octene sulfonyl fluoride, converted to either the proton (H+), or the metal salt form.

A soluble acidic oligomerization catalyst may also be used in the method of the invention. Suitable soluble acids include, those acid catalysts with a pKa less than about 4, preferably with a pKa less than about 2, including inorganic acids, organic sulfonic acids, heteropolyacids, perfluoroalkylsulfonic acids, and combinations thereof. Also suitable are metal salts of acids with pKa less than about 4, including metal sulfonates, metal sulfates, metal trifluoroacetates, metal triflates, and mixtures thereof, including mixtures of salts with their conjugate acids. Specific examples of suitable acids include sulfuric acid, fluorosulfonic acid, phosphoric acid, p-toluenesulfonic acid, benzenesulfonic acid, phosphotungstic acid, phosphomolybdic acid, trifluoromethanesulfonic acid, 1,1,2,2-tetrafluoroethanesulfonic acid, 1,1,1,2,3,3-hexafluoropropanesulfonic acid, bismuth triflate, yttrium triflate, ytterbium triflate, neodymium triflate, lanthanum triflate, scandium triflate, zirconium triflate, and zinc tetrafluoroborate.

For batch reactions, the acidic oligomerization catalyst is preferably used in an amount of from about 0.01% to about 50% by weight of the reactants (although the concentration of acid catalyst may exceed 50% for reactions run in continuous mode using a packed bed reactor). In a particular embodiment, the range is 0.25% to 5% by weight of the reactants unless the reaction is run in continuous mode using a packed bed reactor. For flow reactors, the acid catalyst will be present in amounts that provide WHSV values ranging from about 0.1 hr⁻¹ to 500 hr⁻¹ (e.g., about 0.1, about 0.5, about 1.0, about 2.0, about 5.0, about 10, about 20, about 30, about 40, about 50, about 60, about 70, about 80, about 90, about 100, about 150, about 200, about 250, about 300, about 350, about 400, about 450, or about 500 hr⁻¹).

Other suitable heterogeneous acid catalysts include, for example, acid treated clays, heterogeneous heteropolyacids and sulfated zirconia. The acid catalyst can also be selected from the group consisting of sulfuric acid-treated silica, sulfuric acid-treated silica-alumina, acid-treated titania, acid-treated zirconia, heteropolyacids supported on zirconia, heteropolyacids supported on titania, heteropolyacids supported on alumina, heteropolyacids supported on silica, and combinations thereof. Suitable heterogeneous acid catalysts include those having an H₀ of less than or equal to 2.

In most embodiments of the present invention, the dimerization reaction step is typically carried out using a fixed-bed reactor using any of the oligomerization catalysts described herein. Alternatively, other types of reactors known in the art can be used, such as fluidized bed reactors, batch reactors, catalytic distillation reactors, etc. In a particular embodiment, the oligomerization catalyst is acidic catalyst such as HZSM-5, solid phosphoric acid, or a sulfonic acid resin.

As described above, the feedstock for the dimerization reaction step is obtained from the product of the dehydration reaction step (e.g., obtained after separating the C₄ alkene product from any unreacted isobutanol). If the dehydration reaction is carried out at pressures below about 30 psig, the C₄ alkene product obtained after gas-liquid separation can be compressed to form a C₄ alkene-rich feedstock for the dimerization reaction. Alternatively, if the dehydration reaction is carried out at higher pressures (e.g., about 60 psig or higher) and/or the dehydration product is separated using liquid-liquid separation, the liquid C₄ alkene-rich phase can be used as the feedstock for the dimerization reaction directly (e.g., pumped directly into the dimerization reactor), or can be diluted with suitable diluents as described herein. In particular embodiments, the liquid C₄ alkene-rich feedstock contains unreacted isobutanol from the dehydration reaction, and/or additional diluents added to improve the selectivity of the dimerization reaction step. In most embodiments, the C₄ alkene comprises isobutylene. In typical embodiments, it is desirable that the C₄ alkene portion of the feedstock comprises at least about 95% isobutylene, or at least about 96%, at least about 97%, at least about 98%, at least about 99%, or about 100% isobutylene.

As discussed herein, higher selectivity for formation of dimers such as 2,4,4-trimethylpentenes, 2,5 dimethylhexenes, and 2,5-dimethylhexadienes is favored at lower conversion and under milder oligomerization conditions (e.g., lower temperature and pressure). In most embodiments, the reaction is carried out in the liquid phase at a pressure ranging from 0-1500 psig, and at a temperature of about 250° C. or less. In some embodiments, the oligomerization reaction pressure is about 0, about 15, about 30, about 45, about 60, about 75, about 90, about 105, about 120, about 135, about 150, about 165, about 180, about 195, about 210, about 225, about 240, about 255, about 270, about 285, about 300, about 350, about 400, about 450, about 500, about 550, about 600, about 650, about 700, about 750, about 800, about 850, about 900, about 950, about 1000, about 1100, about 1200, about 1300, about 1400, or about 1500 psig, inclusive of all ranges and subranges therebetween.

In other embodiments, the dimerization reaction temperature is about 250° C. or less, about 225° C. or less, about 200° C. or less, about 175° C. or less, about 150° C. or less, about 125° C. or less, about 100° C. or less, about 75° C. or less, or about 50° C. or less, inclusive of all ranges and subranges therebetween. In a specific embodiment, the oligomerization temperature is about 170° C.

The weight hourly space velocity (WHSV) of the oligomerization reaction can range from about 1 hr⁻¹ to about 500 hr⁻¹, or about 1, about 2, about 3, about 4, about 5, about 6, about 7, about 8, about 9, about 10, about 15, about 20, about 25, about 30, about 35, about 40, about 45, about 50, 55, about 60, about 65, about 70, about 75, about 80, about 85, about 90, about 95, about 100, about 110, about 120, about 130, about 140, about 150, about 175, about 200, about 225, about 250, about 275, about 300, about 350, about 400, about 450, or about 500 hr⁻¹. In a specific embodiment, the WHSV is about 5 hr⁻¹.

The renewable C₈ alkenes prepared after the oligomerization step in the process of the present invention have three, two or at least one double bond. On average, the product of the oligomerizing step of the process of the present invention has less than about two double bonds per molecule, in particular embodiments, less than about 1.5 double bonds per molecule. In most embodiments, the C₈ alkenes have on average one double bond.

Selective dimerization of the C₄ alkene during the dimerization reaction step can also be provided by the addition of alcohols such as t-butanol and diluents such as paraffins (such as kerosene, isooctane, or isobutane) to the oligomerization feedstock. In other embodiments, the selectivity of the dimerization reaction can be enhanced by adding water and isobutanol, e.g., by adding aqueous isobutanol, or by incompletely drying the C₄ alkene (isobutylene) product obtained from the dehydration reaction step (which contains unreacted isobutanol).

Some rearrangement of the C₄ alkene feedstock or C₈ alkene product may also occur during dimerization, thereby introducing new or undesired branching patterns into the C₈ alkene products. In most embodiments, rearrangement of the C₄ alkene feedstock and/or C₈ alkene product is not desirable, particularly when the oligomerization feedstock is isobutylene, and/or the oligomerization product is a 2,4,4-trimethylpentene, 2,5-dimethylhexene, or 2,5-dimethylhexadiene. In such embodiments, the reaction conditions and catalyst are selected to minimize or eliminate rearrangement (e.g., temperatures below at least about 200° C., or below about 180° C., and in particular embodiments, about 170° C.). In other embodiments, where the C₄ alkene feedstock includes some amount of unbranched C₄ alkene (i.e., 1-butene or 2-butene), the dimerization reaction could be carried out under conditions which favor dimerization and rearrangement to branched dimers such as 2,4,4-trimethylpentenes, 2,5-dimethylhexenes, or 2,5-dimethylhexadienes or under conditions in which linear butenes do not dimerize (or dimerize at a substantially lower rate compared to isobutylene), thereby maximizing the selectivity of the dimerization for 2,4,4-trimethylpentenes. Alternatively, the linear butenes could be isomerized by recycling the linear butenes to a separate isomerization reactor, after which the isomerized product (e.g., isobutylene) is then added back to the dimerization feedstock. Linear butene isomers can also be collected for use as a feedstock for other processes (for example, oligomerization to predominantly unbranched higher molecular weight hydrocarbons suitable for use as e.g. diesel fuel).

Similarly, if the C₈ alkene dimerization product is unbranched or includes C₈ isomers which do not dehydrocyclize selectively to p-xylene, it may be desirable to promote rearrangement of the dimerization feedstock to isobutylene and/or the dimerization product to 2,4,4-trimethylpentenes, 2,5-dimethylhexenes, or 2,5-dimethylhexadienes. Rearrangement to more desirable branched isomers (e.g., 2,4,4-trimethylpentenes, 2,5-dimethylhexenes, or 2,5-dimethylhexadienes) can be promoted by dimerization at lower temperatures and/or at higher WHSV values, or the less desirable C₈ alkene isomers can be isomerized by recycling back to the dimerization reactor, or by recycling to a separate isomerization reactor, after which the isomerized product (e.g., 2,4,4-trimethylpentenes, 2,5-dimethylhexenes, or 2,5-dimethylhexadienes) is then added to the dehydrocyclization feedstock.

As discussed above, p-xylene (and other aromatics) are currently produced by catalytic cracking and catalytic reforming of petroleum-derived feedstocks. In particular, the catalytic reforming process uses light hydrocarbon “cuts” like liquefied petroleum gas (C₃ and C₄) or light naphtha (especially C₅ and C₆), which are then converted to C₆-C₈ aromatics, typically by one of the three main petrochemical processes such as M-2 Forming (Mobil), Cyclar (UOP) and Aroforming (IFP-Salutec). These processes use new catalysts which were developed to produce petrochemical grade benzene, toluene, and xylene (BTX) from low molecular weight alkanes in a single step. The process can be described as “dehydrogenation and dehydrocyclooligomerization” over one catalyst and in single reaction zone (the use of C₃ hydrocarbons requires oligomerization rather than dimerization to prepare substituted aromatics).

A variety of alumina and silica based catalysts and reactor configurations have been used to prepare aromatics from low molecular weight hydrocarbons. For example, the Cyclar process developed by UOP and BP for converting liquefied petroleum gas into aromatic compounds uses a gallium-doped zeolite (Appl. Catal. A, 1992, 89, p. 1-30). Other reported catalysts include bismuth, lead, or antimony oxides (U.S. Pat. No. 3,644,550 and U.S. Pat. No. 3,830,866), chromium treated alumina (U.S. Pat. No. 3,836,603 and U.S. Pat. No. 6,600,081), rhenium treated alumina (U.S. Pat. No. 4,229,320) and platinum treated zeolites (WO 2005/065393 A2). A non-limiting list of such catalysts include mixtures of chromia-alumina and bismuth oxide (e.g., bismuth oxide prepared by the thermal decomposition of bismuth compounds such as bismuth nitrate, bismuth carbonate, bismuth hydroxide, bismuth acetate, etc. and e.g., chromia-alumina prepared by impregnating alumina particles with a chromium composition to provide particles containing about 5, about 10, about 15, about 20, about 25, about 30, about 35, about 40, about 45, or about 50 mol % chromia, optionally including a promoter such as potassium, sodium, or silicon, and optionally including a diluent such as silicon carbide, α-alumina, zirconium oxide, etc.); bismuth oxide, lead oxide or antimony oxide in combination with supported platinum, supported palladium, supported cobalt, or a metal oxide or mixtures thereof, such as chromia-alumina, cobalt molybdate, tin oxide or zinc oxide; supported chromium on a refractory inorganic oxide such as alumina or zirconia, promoted with metal such as iron, tin, tungsten, optionally in combination with a Group I or II metal such as Na, K, Rb, Cs, Mg, Ca, Sr, and Ba); rhenium in oxide or metallic form deposited on a neutral or weakly acidic support which has been additionally impregnated with an alkali metal hydroxide or stannate and subsequently reduced with hydrogen at elevated temperatures; and platinum deposited on aluminosilicate MFI zeolite. Any of these known catalysts can be used in the process of the present invention. In particular embodiments of the process of the present invention, the dehydrocyclization catalyst includes, for example, chromium-oxide treated alumina, platinum- and tin-containing zeolites and alumina, cobalt- and molybdenum-containing alumina, etc. In a specific embodiment, the dehydrocyclization catalyst is a commercial catalyst based on chromium oxide on an alumina support.

High selectivity for p-xylene in the dehydrocyclization reaction is favored by providing a dehydrocyclization feedstock which comprises primarily 2,4,4-trimethylpentenes, 2,5-dimethlyhexenes, and/or 2,5-dimethylhexadienes by appropriate selection of dehydrocyclization catalyst (as described herein), and by appropriate selection of dehydrocyclization process conditions (e.g., process temperature, pressure, WHSV, etc.). In most embodiments, the dehydrocyclization reaction is carried out below or slightly above atmospheric pressure, for example at pressures ranging from about 1 psia to about 20 psia, or about 1 psia, about 2 psia, about 3 psia, about 4 psia, about 5 psia, about 6 psia, about 7 psia, about 8 psia, about 9 psia, about 10 psia, about 11 psia, about 12 psia, about 13 psia, about 14 psia, about 15 psia, about 16 psia, about 17 psia, about 18 psia, about 19 psia, and about 20 psia, inclusive of all ranges and subranges therebetween. In most embodiments, the dehydrocyclization is carried out at temperatures ranging from about 400° C. to about 600° C., or about 400° C., about 425° C., about 450° C., about 475° C., about 500° C., about 525° C., about 550° C., about 575° C., and about 600° C., inclusive of all ranges and subranges therebetween. In most embodiments, the dehydrocyclization is carried out at WHSV values of about 1 hr⁻¹, for example about 0.51 hr⁻¹, about 1 hr⁻¹, about 1.5 hr⁻¹, or about 2 hr⁻¹, inclusive of all ranges and subranges therebetween. In most embodiments, the dehydrocyclization reaction is operated at conversions ranging from about 20-50%, and provides a p-xylene selectivity (i.e., the percentage of xylene products which is p-xylene) greater than about 75%. In other embodiments, the p-xylene selectivity is ≧about 75%, ≧about 80%, ≧about 85%, ≧about 90%, ≧about 95%, ≧about 96%, ≧about 97%, ≧about 98%, or ≧about 99%.

In addition, both the conversion and selectivity of the dehydrocyclization reaction for p-xylene can be enhanced by adding diluents to the feedstock, such as hydrogen, nitrogen, argon, and methane. Unreacted C₄ alkene (e.g. isobutylene from the oligomerization reaction) can also be used as an effective diluent to improve the p-xylene selectivity of the dehydrocyclization reaction, and to help suppress cracking. Accordingly, in some embodiments, the selectivity of the dimerization reaction step is improved by carrying out the dimerization under low conversion conditions, as discussed above, such that the product from the dimerization reaction contains significant amounts of unreacted C₄ alkene (e.g., isobutylene), a portion of which can be recycled back to the dimerization reaction feedstock, and a portion of which is present in the dehydrocyclization reaction feedstock. Any C₄ alkene (or C₄ alkane) remaining in the product of the dehydrocyclization reaction can then be recycled back into the dimerization feedstock and/or the dehydrocyclization feedstock. In some embodiments, the dehydrocyclization feedstock comprises 1-100% 2,4,4-trimethylpentenes, 2,5-dimethlyhexenes, and/or 2,5-dimethylhexadienes, with the balance diluent. In particular embodiments, the dehydrocyclization feedstock comprises less than about 50% 2,4,4-trimethylpentenes, 2,5-dimethlyhexenes, and/or 2,5-dimethylhexadienes to reduce “coking” of the dehydrocyclization catalyst. For example, the dehydrocyclization feedstock comprises about 1%, about 2%, about 5%, about 10%, about 15% about 20%, about 25%, about 30%, about 35%, about 40%, about 45%, or about 50% 2,4,4-trimethylpentenes, 2,5-dimethlyhexenes, and/or 2,5-dimethylhexadienes, inclusive of all ranges and sub-ranges therebetween.

The conversion of alkenes and alkanes into aromatic compounds is a net oxidation reaction that releases hydrogen from the aliphatic hydrocarbons. If no oxygen is present, hydrogen gas is a co-product, and light alkanes such as methane and ethane are by-products. If oxygen is present, the hydrogen is converted into water. The dehydrocyclization reaction step of the present invention is typically carried out in the relative absence of oxygen (although trace levels of oxygen may be present due to leaks in the reactor system, and/or the feedstock for the dehydrocyclization reaction step may have trace contamination with oxygen). The hydrogen and light hydrocarbons produced as a by-product of the dehydrocyclization reaction are themselves valuable compounds that can be removed and used for other chemical processes (e.g., hydrogenation of alkene by-products, for example C₈ alkenes such as 2,4,4-trimethylpentenes) to produce alkanes suitable for use as renewable fuels or renewable fuel additives (e.g., isooctane), etc.) in analogy to the practice in traditional petrochemical refineries that produces aromatics, these light compounds are collected and used throughout the refinery. This hydrogen also reacts with isobutylene and diisobutylene to produce isobutane and isooctane which can be recycled to use as diluents for oligomerization (isobutane and isooctane) or feedstock for dehydrocyclization to form isobutylene by dehydrogenation of isobutane and p-xylene by dehydrocyclization of isooctane. The mixture of hydrogen and light hydrocarbons produced from the dehydrocyclization reaction can be used for hydrogenation without further purification, or the light hydrocarbons can be removed (either essentially completely or a portion thereof) to provide relatively pure or higher purity hydrogen prior to the hydrogenation reaction.

Hydrogenation is carried out in the presence of a suitable active metal hydrogenation catalyst. Acceptable solvents, catalysts, apparatus, and procedures for hydrogenation in general can be found in Augustine, Heterogeneous Catalysis for the Synthetic Chemist, Marcel Decker, New York, N.Y. (1996).

Many hydrogenation catalysts known in the art are effective, including (without limitation) those containing as the principal component iridium, palladium, rhodium, nickel, ruthenium, platinum, rhenium, compounds thereof, combinations thereof, and the supported versions thereof.

Typically, the high temperatures at which these dehydrocyclization reactions are carried out tend to coke up and deactivate the catalysts. To reuse the catalyst, the coke must be removed as frequently as every 15 minutes, usually by burning it off in the presence of air. Thus, even though the dehydrocyclization reaction itself is, in most embodiments of the present invention, carried out in the absence of oxygen, oxygen (and optionally hydrogen) can periodically be introduced to reactivate the catalyst. The presence of hydrogenating metals such as nickel, platinum, and palladium in the catalyst will catalyze the hydrogenation of the coke deposits and extend catalyst life. In order to accommodate reactivation of the catalyst in a continuous process, two or more dehydrocyclization reactors can be used so that at least one dehydrocyclization reactor is operational while other dehydrocyclization reactors are taken “off line” in order to reactivate the catalyst. When multiple dehydrocyclization reactors are used, they can be connected in parallel or in series.

As discussed above, the hydrocarbon feedstocks used to form aromatic compounds in conventional petroleum refineries are typically mixtures of hydrocarbons. As a result, the p-xylene produced by petroleum refineries is mixed with other xylene isomers and other aromatics (e.g., light aromatics such as benzene and toluene, as well as ethylbenzene, etc.), requiring further separation and purification steps in order to provide suitably pure p-xylene for subsequent conversion to terephthalic acid or terephthalate esters suitable for polyester production. In a large-scale refinery, producing pure streams of p-xylene can be expensive and difficult. In contrast, the process of the present invention can readily provide relatively pure, renewable p-xylene at a cost which is competitive with that of petroleum derived p-xylene from conventional refineries.

For example, a biomass derived C₄ alcohol (e.g. aqueous isobutanol from fermentation) is dehydrated in the vapor phase over an acidic dehydration catalyst (e.g., gamma alumina) to form a product containing unreacted C₄ alcohol and 99% isobutylene (based on the total amount of olefin product). Isobutylene is removed from the dehydration product stream in the vapor phase from a condensed water/C₄ alcohol phase using e.g., a gas/liquid separator. Unreacted C₄ alcohol is recycled back into the dehydration reaction feedstock. Condensed isobutylene is then oligomerized to form diisobutylene (e.g., ≧about 95% 2,4,4-trimethylpentenes) at about 50% conversion in an oligomerization reactor containing a metal-doped zeolite catalyst (e.g., HZSM-5). A portion of the unreacted isobutylene is recycled back to the oligomerization feedstock, while a remaining portion of the isobutylene remains in the product stream to serve as a diluent in the subsequent dehydrocyclization reaction step. The resulting mixture of diisobutylene and isobutylene, and optionally additional diluent (e.g., hydrogen, nitrogen, argon, and methane) is then fed into a dehydrocyclization reactor and reacted in the presence of a dehydrocyclization catalyst to selectively form p-xylene (e.g., >95% of the xylenes is p-xylene). Hydrogen produced as a co-product of the dehydrocyclization can be recycled back to the dehydrocyclization feedstock as a diluent, or alternatively used as a reactant to produce other compounds (e.g., to hydrogenate alkenes or alkene by-products for use as fuels or fuel additives, e.g., hydrogenate C₈ olefins such as isooctene to make isooctane for transportation fuels). Light alkanes in the hydrogen can be separated out before the purified hydrogen is utilized, or the impure light alkane/hydrogen mixture can be used directly in hydrogenation reactions. Unreacted isobutylene can be recycled back to the oligomerization feedstock, and/or fed to the dehydrocyclization feedstock as a diluent.

The resulting high purity p-xylene can be condensed from the product stream of the dehydrocyclization reaction and converted to terephthalic acid (TPA) or terephthalate esters (TPA esters) without further purification. However, since the purity requirements for TPA or TPA esters used as monomers in preparing PET is quite high (e.g., typically >about 99.5% purity), it may be desirable to further purify the renewable p-xylene prepared by the process of the present invention, e.g. by known methods such as simulated moving bed chromatography, fractional crystallization or fractional distillation. Although such methods are used in conventional petrochemical process for preparing high purity p-xylene, the “crude” p-xylene produced from the conventional process contains substantial amounts of impurities and undesirable xylene isomers (˜10-30% impurities) and typically requires multiple purification steps to obtain the required purity level. In contrast, the “crude” p-xylene prepared by the process of the present invention is substantially more pure than conventional petrochemically produced p-xylene, and requires only minimal purification, if at all, to obtain purities suitable for preparing TPA or TPA ester monomers for polyester production.

p-Xylene is converted into either TPA or TPA esters by oxidation over a transition metal-containing catalyst (Ind. Eng. Chem. Res. 2000, 39, p. 3958-3997 reviews the patent literature). Dimethyl terephthalate (DMT) has been traditionally produced at higher purity than TPA, and can be used to manufacture PET as well. Methods for producing TPA and DMT are taught in U.S. Pat. Nos. 2,813,119; 3,513,193; 3,887,612; 3,850,981; 4,096,340; 4,241,220; 4,329,493; 4,342,876; 4,642,369; and 4,908,471. TPA can be produced by oxidizing p-xylene in air or oxygen (or air or oxygen diluted with other gases) over a catalyst containing manganese and cobalt, although nickel catalysts have also been used with some success. Acetic acid is used as a solvent for these oxidation reactions and a bromide source such as hydrogen bromide, bromine, or tetrabromoethane is added to encourage oxidation of both methyl groups of the xylene molecule with a minimum of by-products. The temperatures of the reactions are generally kept between 80-270° C. with residence times of a few hours. The TPA is insoluble in acetic acid at lower temperatures (i.e. below 100° C.), which is how it is separated and purified. DMT can be produced by esterification of the “crude” product of the TPA reactions described above with methanol, and purification by distillation. A single step process to produce DMT by oxidizing p-xylene in the presence of methanol was developed by DuPont but is not often used due to low yields. All of these processes also produce monomethylesters of TPA which can be hydrolyzed to form the TPA or further esterified to form the diester, e.g., DMT.

Polyesters such as PET (polyethylene terephthalate) are prepared by polymerizing ethylene glycol with TPA or TPA esters, and thus 80% of the carbon content of PET resides in the terephthalate moiety of the polymer. Accordingly, PET prepared from renewable TPA or TPA esters, prepared as described herein, would comprise at least 80% renewable carbon. A completely renewable PET can be prepared by polymerizing TPA or TPA esters prepared according to the methods of the present invention with renewable ethylene glycol, prepared e.g. by the method of Mazloom et al., Iranian Polymer Journal, 16(9), 2007, 587-596; or Schonnagle et al., EP 1447506 A1.

Other renewable polymers, for example polyesters such as PTT (polytrimethylene terephthalate) or PBT (polybutylene terephthalate) can also be prepared from the renewable TPA or TPA esters as described herein by reaction of renewable TPA or TPA esters with any appropriate comonomer (e.g., 1,3-propylene glycol, butylene glycol, etc.) or other comonomers (polyols, polyamines, etc.) which react with TPA or TPA esters.

The processes of the present invention provide renewable p-xylene, which is environmentally advantageous compared to conventional processes for preparing p-xylene from petrochemical feedstock. In addition, the processes of the present invention are highly selective in forming p-xylene, whereas conventional petrochemical processes for preparing p-xylene are relatively nonselective overall. Conventional petrochemical processes for preparing high purity p-xylene are relatively nonselective and provide a mixture of aromatic compounds, from which the p-xylene must be isolated and purified to a level suitable for e.g., production of terephthalic acid. In addition, conventional petrochemical processes for preparing p-xylene often include unit operations for separating p-xylene from by-products such as benzene, toluene, ethylbenzene, and/or for converting such by-products to xylenes (including p-xylene), and/or for isomerizing o- and m-xylenes to p-xylene. In contrast, in various embodiments of the present invention can directly provide p-xylene of sufficient purity that such purification, conversion, and isomerization steps are generally not required. That is, in most embodiments, the processes of the present invention do not include steps of separating p-xylene from other xylene isomers, or separating p-xylene from other aromatic by-products (such as those described herein), or isomerizing by-product C₈ aromatics to p-xylene. In other embodiments, only minimal purification of the p-xylene is required (e.g., by separating the p-xylene from other xylene isomers or aromatic by-products).

The conversion of isooctene to p-xylene requires that typical multi-branched isooctene isomers such as 2,4,4-trimethylpentene are converted to 2,5-dimethylhexadiene before subsequent cyclization and dehydrogenation to p-xylene. When 2,5-dimethylhexadiene is reacted over the dehydrocyclization catalysts used to convert 2,4,4-trimethylpentenes to p-xylene, the 2,5-dimethylhexadiene is quantitatively converted into p-xylene whereas 2,4,4-trimethylpentene is at best only converted to p-xylene in 50% yield. To explain this fact, Anders, et al. (Chemische Technik 1986, 38, 116-119) propose a thermally catalyzed radical decomposition mechanism of 2,4,4-trimethylpentene which converts 2 equivalents of 2,4,4-trimethylpentene to 1 equivalent of 2,5-dimethylhexadiene and 2 equivalents of isobutane/isobutylene before conversion to p-xylene occurs under dehydrocyclization conditions. The isobutane/isobutylene produced from the reaction can be recycled to produce additional isooctene. To obtain high single pass yields from an isobutylene dimer, however, it is desired to first convert isobutylene directly to 2,5-dimethylhexadiene or 2,5-dimethylhexene then to pass the dimethylhexadiene or dimethylhexene over the dehydrocyclization catalyst to produce p-xylene in >50% yield. In the absence of oxygen, isobutylene is dimerized to 2,5-dimethylhexene over transition metal catalysts such as palladium(III) chloride or rhodium(III) chloride (e.g. French Patent 1499833A), cobalt(II) acetylacetonate and triethylaluminum (e.g. U.S. Pat. No. 5,320,993), or nickel with phosphorous and nitrogen chelating ligands (e.g. Journal of Catalysis 2004, 226, 235-239). Alternatively, dimerization/dehydrogenation of isobutylene to 2,5-dimethylhexadiene occurs in the presence of oxygen and a metal oxide catalyst, although at much lower yields than non-oxygenated processes. Multiple types of metal oxide and other metal catalysts including oxides, phosphides, and alloys of bismuth, tin, indium, thallium, antimony, cadmium, copper, iron, palladium, tungsten, niobium, arsenic, and niobium are used to dehydrodimerize olefins (e.g. Catalysis Today 1992, 14, 343-393). Both 2,5-dimethylhexadiene and 2,5-dimethylhexene are converted to p-xylene under the dehydrocyclization conditions described for 2,4,4-trimethylpentene with 2,5-dimethylhexene producing less hydrogen than the equivalent diene. In addition, the oxidative dehydrodimerization catalyst can be combined with a cyclizing catalyst (e.g., platinum on aluminum oxide, chromium on aluminum oxide, etc.) to increase the selectivity for cyclization to p-xylene. When the isobutylene converted to dimethylhexadiene or dimethylhexene is derived from renewable isobutanol, renewable p-xylene is obtained in high yield.

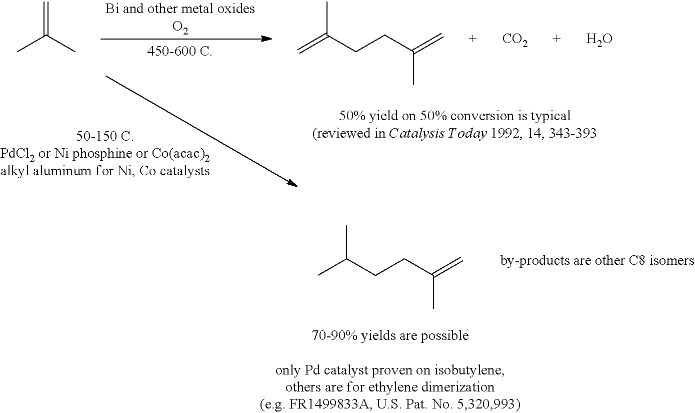

As discussed herein, the dimerization of C₄ alkenes to C₈ alkenes, and subsequent cyclodehydration to p-xylene can be carried out in a step-wise fashion, in which the dimerization product (comprising e.g., 2,4,4-trimethylpentenes, 2,5-dimethylhexenes, and/or 2,5-dimethylhexadienes) is isolated and optionally purified prior to cyclodehydration to p-xylene, or passed directly to the cyclodehydration reactor (or reaction zone) without isolation or purification. Alternatively, by appropriate selection of reaction conditions (i.e., catalyst(s), reaction temperature and pressure, reactor design, etc.) the dimerization and cyclodehydration reactions can be carried out essentially simultaneously, such that the C₄ alkene is effectively converted directly to p-xylene. In this regard, “essentially simultaneous” reaction steps could include direct conversion of the C₄ alkene (e.g., isobutylene) to p-xylene in a single reaction step, or rapid sequential conversion of the C₄ alkene to an intermediate (e.g., a C₈ alkene or other intermediate), which under the reaction conditions is rapidly converted to p-xylene such that no intermediates are isolated (or need be isolated).

For example, conversion of isobutylene directly to p-xylene can be carried out using a bismuth oxide catalyst under oxidative conditions, as described above, or alternatively reacting isobutylene, prepared as described herein, using conditions and catalysts used in petrochemical processes such as the M-2 Forming process (Mobil), Cyclar process (UOP) and Aroforming process (IFP-Salutec), to form an aromatic product comprising p-xylene.

### EXAMPLES

### Example 1

An overnight culture was started in a 250 mL Erlenmeyer flask with microorganism from a freezer stock (e.g., Escherichia coli modified to produce isobutanol, e.g., the organism described in U.S. Ser. No. 12/263,436) with a 40 mL volume of modified M9 medium consisting of 85 g/L glucose, 20 g/L yeast extract, 20 μM ferric citrate, 5.72 mg/L H₃BO₃, 3.62 mg/L MnCl₂.4H₂O, 0.444 mg/L ZnSO₄.7H₂O, 0.78 mg/L Na₂MnO₄.2H₂O, 0.158 mg/L CuSO₄.5H₂O, 0.0988 mg/L CoCl₂.6H₂O, 6.0 g/L NaHPO₄, 3.0 g/L KH₂PO₄, 0.5 g/L NaCl, 2.0 g/L NH₄Cl, 0.0444 g/L MgSO₄, and 0.00481 g/L CaCl₂ and at a culture OD₆₀₀ of 0.02 to 0.05. The starter culture was grown for approximately 14 hrs in a 30° C. shaker at 250 rpm. Some of the starter culture was then transferred to a 400 mL DasGip fermentor vessel containing about 200 mL of modified M9 medium to achieve an initial culture OD₆₀₀ of about 0.1. The vessel was attached to a computer control system to monitor and control the fermentation to a pH of 6.5 (by appropriate addition of base), a temperature of 30° C., dissolved oxygen levels, and agitation. The vessel was agitated, with a minimum agitation of 200 rpm—the agitation was varied to maintain a dissolved oxygen content of about 50% of saturation using a 12 sl/h air sparge until the OD₆₀₀ was about 1.0. The vessel was then induced with 0.1 mM IPTG. After continuing growth for approximately 8-10 hrs, the dissolved oxygen content was decreased to 5% of saturation with 200 rpm minimum agitation and 2.5 sl/h airflow. Continuous measurement of the fermentor vessel off-gas by GC-MS analysis was performed for oxygen, isobutanol, ethanol, carbon dioxide, and nitrogen throughout the experiment. Samples were aseptically removed from the fermentor vessel throughout the fermentation and used to measure OD₆₀₀, glucose concentration, and isobutanol concentration in the broth. Isobutanol production reached a maximum at around 21.5 hrs with a titer of 18 g/L and a yield of approximately 70% maximum theoretical. The broth was subjected to vacuum distillation to provide a 84:16 isobutanol/water mixture which was redistilled as needed to provide dry isobutanol.

### Example 2

GEVO1780 is a modified bacterial biocatalyst (described in U.S. Publ. No. 2009/0226990) that contains genes on two plasmids which encode a pathway of enzymes that convert pyruvate into isobutanol. When the biocatalyst GEVO1780 was contacted with glucose in a medium suitable for growth of the biocatalyst, at about 30° C., the biocatalyst produced isobutanol from the glucose. An overnight starter culture was started in a 250 mL Erlenmeyer flask with GEVO1780 cells from a freezer stock with a 40 mL volume of modified M9 medium consisting of 85 g/L glucose, 20 g/L yeast extract, 20 μM ferric citrate, 5.72 mg/L H₃BO₃, 3.62 mg/L MnCl₂.4H₂O, 0.444 mg/L ZnSO₄.7H₂O, 0.78 mg/L Na₂MnO₄.2H₂O, 0.158 mg/L CuSO₄.5H₂O, 0.0988 mg/L CoCl₂.6H₂O, NaHPO₄ 6.0 g/L, KH₂PO₄ 3.0 g/L, NaCl 0.5 g/L, NH₄Cl 2.0 g/L, MgSO₄ 0.0444 g/L and CaCl₂ 0.00481 g/L and at a culture OD₆₀₀ of 0.02 to 0.05. The starter culture was grown for approximately 14 hrs in a 30° C. shaker at 250 rpm. Some of the starter culture was then transferred to a 2000 mL DasGip fermenter vessel containing about 1500 mL of modified M9 medium to achieve an initial culture OD₆₀₀ of about 0.1. The vessel was attached to a computer control system to monitor and control pH at 6.5 through addition of base, temperature at about 30° C., dissolved oxygen, and agitation. The vessel was agitated, with a minimum agitation of 400 rpm and agitation was varied to maintain a dissolved oxygen content of about 50% using a 25 sL/h air sparge until the OD₆₀₀ was about 1.0. The vessel was then induced with 0.1 mM IPTG. After continuing growth for approximately 8-10 hrs, the dissolved oxygen content was decreased to 5% with 400 rpm minimum agitation and 10 sl/h airflow. Continuous measurement of the fermentor vessel off-gas by GC-MS analysis was performed for oxygen, isobutanol, ethanol, and carbon dioxide throughout the experiment. Samples were aseptically removed from the fermenter vessel throughout the experiment and used to measure OD₆₀₀, glucose concentration, and isobutanol concentration in the broth. Throughout the experiment, supplements of pre-grown and pre-induced biocatalyst cells were added as a concentrate two times after the start of the experiment: at 40 h and 75 h. These cells were the same strain and plasmids indicated above and used in the fermenter. Supplemented cells were grown as 1 L cultures in 2.8 L Fernbach flasks and incubated at 30° C., 250 RPM in Modified M9 Medium with 85 g/L glucose. Cultures were induced upon inoculation with 0.1 mM IPTG. When the cells had reached an OD₆₀₀ of about 4.0-5.0, the culture was concentrated by centrifugation and then added to the fermenter. A glucose feed of about 500 g/L glucose in DI water was used intermittently during the production phase of the experiment at time points greater than 12 h to maintain glucose concentration in the fermenter of about 30 g/L or above.

The fermenter vessel was attached by tubing to a smaller 400 mL fermenter vessel that served as a flash tank and operated in a recirculation loop with the fermenter. The biocatalyst cells within the fermenter vessel were isolated from the flash tank by means of a cross-flow filter placed in-line with the fermenter/flash tank recirculation loop. The filter only allowed cell-free fermentation broth to flow from the fermenter vessel into the flash tank. The volume in the flash tank was approximately 100 mL and the hydraulic retention time was about 10 minutes. Heat and vacuum were applied to the flash tank. The vacuum level applied to the flash tank was initially set at about 50 mBar and the flash tank was set at about 45° C. These parameters were adjusted to maintain approximately 6-13 g/L isobutanol in the fermenter throughout the experiment. Generally, the vacuum ranged from 45-100 mBar and the flash tank temperature ranged from 43° C. to 45° C. throughout the experiment. Vapor from the heated flash tank was condensed into a collection vessel as distillate. Cell-free fermentation broth was continuously returned from the flash tank to the fermentation vessel.

The distillate recovered in the experiment was strongly enriched for isobutanol. Isobutanol formed an azeotrope with water and usually lead to a two phase distillate: an isobutanol rich top phase and an isobutanol lean bottom phase. Distillate samples were analyzed by GC for isobutanol concentration. Isobutanol production reached a maximum at around 118 hrs with a total titer of about 87 g/L. The isobutanol production rate was about 0.74 g/L/h on average over the course of the experiment. The percent theoretical yield of isobutanol was approximately 90.4% at the end of the experiment. The broth was subjected to vacuum distillation to provide a 84:16 isobutanol/water mixture which was redistilled as needed to provide dry isobutanol.

### Example 3

### Dry Isobutanol Dehydration

Dry isobutanol (<1 wt % water) obtained in Example 2 was fed through a preheater to a fixed-bed tubular reactor packed with a commercial γ-alumina dehydration catalyst (BASF AL-3996). The internal reactor temperature was maintained at 325° C. and the reactor pressure was atmospheric. The WHSV of the isobutanol was 5 hr⁻¹. Primarily isobutylene and water were produced in the reactor, and were separated in a gas-liquid separator at 20° C.; the water had <1% of unreacted isobutanol and the conversion was >99.8%. GC-FID analysis of the gas phase effluent indicated it was 95% isobutylene, 3.5% 2-butene (cis and trans) and 1.5% 1-butene.

### Example 4

### Wet Isobutanol Dehydration

Wet isobutanol (containing 15% water) obtained in Example 2 was fed through a preheater to a fixed-bed tubular reactor packed with a commercial dehydration catalyst (BASF AL-3996). The internal reactor temperature was maintained at 275° C. and the reactor pressure was atmospheric. The WHSV of the isobutanol was 10 hr⁻¹. Primarily isobutylene and water were produced in the reactor, and were separated in a gas-liquid separator at 20° C.; two liquid phases were recovered: one phase comprised water saturated with isobutanol and the other isobutanol-rich phase comprised isobutanol saturated with water. The isobutanol-rich phase was approximately 70% of the liquid effluent, indicating that isobutanol conversion in the reactor was approximately 40%. GC-FID analysis of the gas phase effluent indicated it was about 99% isobutylene, about 0.6% 2-butene (cis and trans) and about 0.4% 1-butene.

### Example 5

### Dry Isobutanol Dehydration at 60 psig

Dry isobutanol (<1 wt % water) obtained in Example 2 was fed through a preheater to a fixed-bed tubular reactor packed with a commercial γ-alumina dehydration catalyst (BASF AL-3996). The internal reactor temperature was maintained at 325° C. and the reactor pressure was maintained at 60 psig. The WHSV of the isobutanol was 5 hr⁻¹. Primarily isobutylene and water were produced in the reactor, and were separated in a liquid-liquid separator at 20° C.; the water had <1% of unreacted isobutanol and the conversion was >99.8%. GC-FID analysis of the gas phase effluent indicated it was 95% isobutylene, 3.5% 2-butene (cis and trans) and 1.5% 1-butene.

### Example 6

### Dry n-Butanol Dehydration at 60 psig

Dry n-butanol (<1 wt % water) is fed through a preheater to a fixed-bed tubular reactor packed with a commercial γ-alumina dehydration catalyst (BASF AL-3996). The internal reactor temperature is maintained at 450° C. and the reactor pressure is maintained at 60 psig. The WHSV of the isobutanol is 3 hr⁻¹. An equilibrium mixture of C₄ olefins and water are produced in the reactor, and are separated in a liquid-liquid separator at 20° C.; the water has <1% of unreacted isobutanol and the conversion is >99.8%. GC-FID analysis of the gas phase effluent indicates it is about 47% isobutylene, about 41% 2-butene (cis and trans) and about 12% 1-butene.

### Example 7

### Oligomerization of Isobutylene

The product stream from Example 3 was dried over molecular sieves, compressed to 60 psig, cooled to 20° C. so that the isobutylene was condensed to a liquid and pumped with a positive displacement pump into a fixed-bed oligomerization reactor packed with a commercial ZSM-5 catalyst (CBV 2314). The reactor was maintained at 175° C. and a pressure of 750 psig. The WHSV of the isobutylene-rich stream was 15 hr⁻¹. The reactor effluent stream was 10% unreacted butenes, 60% isooctenes (primarily 2,4,4-trimethylpentenes), 28% trimers, and 2% tetramers.

### Example 8

### Oligomerization of Isobutylene

The product stream from Example 5 (which was saturated with water) was pumped with a positive displacement pump into a fixed-bed oligomerization reactor packed with a commercial ZSM-5 catalyst (CBV 2314). The reactor was maintained at 170° C. and a pressure of 750 psig. The WHSV of the isobutylene-rich stream was 50 hr⁻¹. The reactor effluent stream was 20% unreacted butenes, 64% isooctenes (primarily 2,4,4-trimethylpentenes), 15% trimers, and 1% tetramers.

### Example 9

### Oligomerization of Isobutylene with Modifier

The product stream from Example 5 is co-fed with 2% wet isobutanol (by weight) and pumped with a positive displacement pump into a fixed-bed oligomerization reactor packed with a commercial ZSM-5 catalyst (CBV 2314). The reactor is maintained at 160° C. and a pressure of 750 psig. The WHSV of the isobutylene-rich stream is 200 hr⁻¹. The product stream is about 30% unreacted butenes, about 69% isooctenes (primarily 2,4,4-trimethylpentenes), and about 1% trimers.

### Example 10

### Oligomerization of Isobutylene with Diluents

The product stream from Example 3 is co-fed with 50% isobutane to a compressor, condensed and pumped into a fixed-bed oligomerization reactor packed with Amberlyst 35 (strongly acidic ionic exchange resin available from Rohm & Haas). The reactor is maintained at 120° C. and a pressure of 500 psig. The WHSV of the isobutylene-rich stream is 100 hr⁻¹. The product stream is about 50% isobutane (diluents), about 3% unreacted butenes, about 44% isooctenes (primarily 2,4,4-trimethylpentenes), and about 3% trimers.

### Example 11

### Oligomerization of Mixed Butenes

The product stream from Example 6 is pumped with a positive displacement pump into a fixed-bed oligomerization reactor packed with a commercial ZSM-5 catalyst (CBV 2314). The reactor is maintained at 170° C. and a pressure of 750 prig. The WHSV of the mixed butene stream is 20 hr⁻¹. The reactor effluent stream is about 60% unreacted butenes (primarily linear butenes), about 36% isooctenes (primarily 2,4,4-trimethylpentenes), and about 4% trimers.

### Example 12

### Recycle of Unreacted Linear Butenes

The product stream from Example 11 is distilled to recover the unreacted butenes (primarily linear butenes). The linear butene-rich stream is condensed and pumped with a positive displacement pump into an isomerization reactor at 450° C. where the equilibrium composition of mixed butenes is re-established. The mixed butene stream is recycled back and combined with the oligomerization reactor feed used in Example 10. The overall system conversion is >99% using the recycle stream and the yield of isooctenes is >89% with approximately 10% trimers.

### Example 13

### Dehydrocyclization of Isooctene

Isooctene from Example 7 was distilled to remove trimers and tetramers and then fed at a molar ratio of 1.3:1 mol nitrogen diluent gas to a fixed bed reactor containing a commercial chromium oxide doped alumina catalyst (BASF D-1145E ⅛″). The reaction was carried out at atmospheric pressure and a temperature of 550° C., with a WHSV of 1.1 hr⁻¹. The reactor product was condensed and analyzed by GC-MS. Of the xylene fraction, p-xylene was produced in greater than 80% selectivity. Analysis by method ASTM D6866-08 showed p-xylene to contain 96% biobased material.

### Example 14

### Dehydrocyclization of Isooctene with Diluents

The product from Example 10 containing 50% isobutane, 3% butenes, 44% isooctenes, and 3% trimers is fed to a fixed bed reactor containing a commercial chromium oxide doped alumina catalyst (BASF D-1145E ⅛″). The reaction is carried out at atmospheric pressure and a temperature of 525° C., with a WHSV of 1.1 hr⁻¹. The reactor product is condensed and analyzed by GC-MS. Of the xylene fraction, p-xylene is produced in greater than 85% selectivity. Hydrogen is also produced and captured for use with other processes.

### Example 15

### Dehydrocyclization of Isooctene with Diluents

Isooctene from Example 8 and diluent isobutylene from Example 5 are fed in a 1:1 molar ratio to a fixed bed reactor containing a commercial chromium oxide doped alumina catalyst (BASF D-1145E ⅛″). The reaction is carried out at atmospheric pressure and a temperature of 550° C., with a WHSV of 1.1 hr⁻¹. The reactor product is condensed and analyzed by GC-MS. Of the xylene fraction, p-xylene is produced in greater than 75% selectivity. Hydrogen is also produced and captured for use with other processes.

### Example 16

### Integrated System to Convert Isobutanol to Renewable p-Xylene

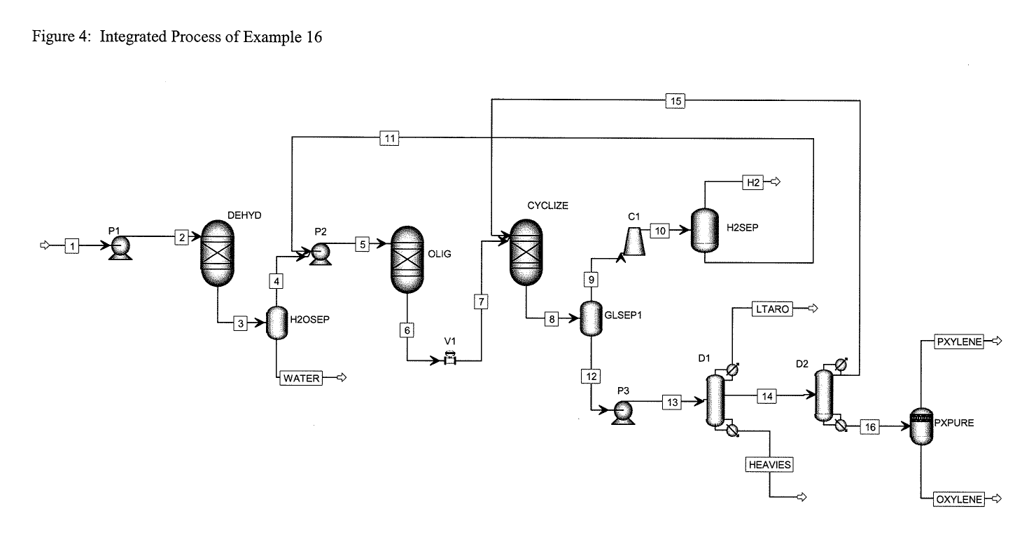

Renewable isobutanol is converted to renewable p-xylene using a process illustrated in FIG. 4. Isobutanol (stream 1) from Example 1 or 2 is fed wet (15 wt % water) through a preheater into a fixed-bed catalyst reactor packed with a commercial γ-alumina catalyst (BASF AL-3996) at a WHSV of 10 hr⁻¹. The dehydration reactor is maintained at 290° C. at a pressure of 60 psig. The effluent (3) from the dehydration reactor is fed to a liquid/liquid separator, where water is removed. Analysis of the organic phase (4) shows that it is 95% isobutylene, 3% linear butenes, and 2% unreacted isobutanol. The organic phase is combined with a recycle stream (11) containing isobutane, isooctane, and unreacted butenes and fed to a positive displacement pump (P2) where it is pumped to an oligomerization reactor packed with HZSM-5 catalyst (CBV 2314) at a WHSV of 100 hr⁻¹. The reactor is maintained at 170° C. at a pressure of 750 psig. The effluent (6) from the oligomerization reactor is analyzed and shown to contain 60% unreacted feed (isobutane, isooctane, and butenes), 39% isooctene, and 1% trimers. The effluent from the oligomerization reactor is combined with recycled isooctene (15) and fed through a preheater and to a fixed bed reactor containing a commercial chromium oxide doped alumina catalyst (BASF D-1145E ⅛″) at a WHSV of 1 hr⁻¹. The dehydrocyclization reactor is maintained at 550° C. and 5 psia. The yield of xylenes from the reactor relative to C₈ alkenes in the feed is 42% with a selectivity to p-xylene of 90%. The effluent (8) is separated with a gas-liquid separator. The gas-phase is compressed (C1) to 60 psig causing the isobutane and butenes to condense. A second gas-liquid separator is used to recover the hydrogen (and small quantities of methane or other light hydrocarbons). The C₄ liquids are recycled (11) and combined with the organic phase from the dehydration reactor (4). The liquid product (12) from the dehydrocyclization reactor is fed to a series of distillation columns slightly above atmospheric pressure by a pump (P3). Any by-product light aromatics (benzene and toluene) and heavy compounds (C₉+ aromatics or isoolefins) are removed. A side stream (14) rich in xylenes and iso-C₈ compounds are fed to a second distillation column. The C₈ compounds (isooctene and isooctane) are recycled (15) to the feed of the dehydrocyclization reactor. The xylene fraction (16) is fed to a purification process resulting in a 99.99% pure p-xylene product and a small byproduct stream rich in o-xylene.

### Example 17

### Oxidation of Renewable p-Xylene to Terephthalic Acid

A 300 mL Parr reactor was charged with glacial acetic acid, bromoacetic acid, cobalt acetate tetrahydrate, and p-xylene, obtained from Example 13, in a 1:0.01:0.025:0.03 mol ratio of glacial acetic acid:bromoacetic acid:cobalt acetate tetrahydrate:p-xylene. The reactor was equipped with a thermocouple, mechanical stirrer, oxygen inlet, condenser, pressure gauge, and pressure relief valve. The reactor was sealed and heated to 150° C. The contents were stirred and oxygen was bubbled through the solution. A pressure of 50-60 psi was maintained in the system and these reaction conditions were maintained for 4 h. After 4 h, the reactor was cooled to room temperature. Terephthalic acid was filtered from solution and washed with fresh glacial acetic acid.

### Example 18

### Purification of Renewable Terephthalic Acid

Terephthalic acid from Example 17 was charged to a 300 mL Parr reactor with 10% Pd on carbon catalyst in a 4.5:1 mol ratio of terephthalic acid: 10% Pd on carbon. Deionized water was charged to the reactor to make a slurry containing 13.5 wt. % terephthalic acid. The reactor was equipped with a thermocouple, mechanical stirrer, nitrogen inlet, hydrogen inlet, pressure gauge, and pressure relief valve. The Parr reactor was sealed and flushed with nitrogen. The Parr reactor was then filled with hydrogen until the pressure inside the reactor reached 600 psi. The reactor was heated to 285° C. and the pressure inside the vessel reached 1000 psi. The contents were stirred under these conditions for 6 h. After 6 h, contents were cooled to room temperature and filtered. The residue was transferred to a vial and N,N-dimethylacetamide was added to the vial in a 5:1 mol ratio of N,N-dimethylacetamide: terephthalic acid. The vial was warmed to 80° C. for 30 minutes to dissolve the terephthalic acid. The contents were filtered immediately; Pd on carbon was effectively removed from the terephthalic acid. Crystallized terephthalic acid filtrate was removed from the collection flask and was transferred to a clean filter where it was washed with fresh N,N-dimethylacetamide and dried. A yield of 60% purified terephthalic acid was obtained.

### Example 19

### Polymerization of Terephthalic Acid to Make Renewable PET

Purified terephthalic acid (PTA) obtained from Example 18 and ethylene glycol are charged to a 300 mL Parr reactor in a 1:0.9 mol ratio of PTA: ethylene glycol. Antimony (III) oxide is charged to the reactor in a 1:0.00015 mol ratio of PTA: antimony (III) oxide. The reactor is equipped with a thermocouple, mechanical stirrer, nitrogen inlet, vacuum inlet, condenser, pressure gauge, and pressure relief valve. The Parr reactor is sealed, flushed with nitrogen, heated to a temperature of 240° C., and pressurized to 4.5 bar with nitrogen. Contents are stirred under these conditions for 3 h. After 3 h, the temperature is increased to 280° C. and the system pressure is reduced to 20-30 mm by connecting the reactor to a vacuum pump. Contents are stirred under these conditions for 3 h. After 3 h, the vacuum valve is closed and the contents of the reactor are flushed with nitrogen. The reactor is opened and contents are immediately poured into cold water to form PET pellets.

### Example 20

### Dimerization of isobutylene to 2,5-dimethylhexenes

The product stream from Example 3 is dried over molecular sieves, compressed to 60 psig, cooled to 20° C. so that the isobutylene is condensed to a liquid, and 100 g is collected. This material is dissolved in 200 mL degassed nitrobenzene under an atmosphere of argon and charged with 10 g of the complex [η²-isobutylene)₂Pd₂Cl₂(μ-Cl)₂] (Kharasch et al., 1938, 60, 882-884 and French Patent 1499833A). After stirring for 2 days 75% of the isobutylene is converted to 1:1 mixture of 2,5-dimethylhex-2-ene and 2,5-dimethylhex-1-ene.

### Example 21

### Dehydrocyclization of 2,5-dimethylhexa-2,4-diene

2,5-dimethylhexa-2,4-diene was run neat through a fixed bed reactor containing a commercial chromium oxide doped alumina catalyst (BASF D-1145E ⅛″). The reaction was carried out at atmospheric pressure and a temperature of 500° C., with a WHSV of 1.0 hr⁻¹. The reactor product was condensed and analyzed by GC-MS. The reactor effluent stream was 60% xylenes, and of the xylene fraction, p-xylene was produced in greater than 99% selectivity.

### Example 22

The product stream from Example 4 is dried over molecular sieves, compressed to 60 psig, cooled to 20° C. so that the isobutylene is condensed to a liquid. The isobutylene is preheated, mixed 4 parts to 1 with molecular oxygen, and then pumped into a ½ inch diameter stainless steel flow reactor packed with particles of 1:1 bismuth:antimony doped with sodium, copper, and zirconium oxides as described in Japan Patent 47-15327 and maintained at a temperature of 420° C. The flow rate of isobutylene over the catalyst in the reactor provides a catalyst contact time of ˜0.45 seconds. The conversion of isobutylene is 32% with 65% selectivity towards diolefin isomers of 2,5-dimethylhexadiene.

### Example 23

The 2,5-dimethylhexadiene product from Example 22 is purified by distillation and is run neat through a fixed bed reactor containing a commercial chromium oxide doped alumina catalyst (BASF D-1145E ⅛″). The reaction is carried out at atmospheric pressure and a temperature of 500° C., with a WHSV of 1.0 hr⁻¹. The reactor product is condensed and analyzed by GC-MS. The reactor effluent stream is 60% xylenes, and of the xylene fraction, p-xylene is produced with greater than 99% selectivity.

### Example 24

The 2,5-dimethylhexene product from Example 21 is purified by distillation and is run neat through a fixed bed reactor containing a commercial chromium oxide doped alumina catalyst (BASF D-1145E ⅛″). The reaction is carried out at atmospheric pressure and a temperature of 500° C., with a WHSV of 1.0 hr⁻¹. The reactor product is condensed and analyzed by GC-MS. The reactor effluent stream is 60% xylenes, and of the xylene fraction, p-xylene is produced with greater than 99% selectivity.

We claim:

1. A method for preparing renewable p-xylene comprising:
(a) treating biomass to form a fermentation feedstock;
(b) fermenting the fermentation feedstock with one or more species of microorganism to form a fermentation broth comprising aqueous isobutanol;
(c) removing aqueous isobutanol from the fermentation broth;
(d) dehydrating, in the presence of a dehydration catalyst, at least a portion of the aqueous isobutanol of step (c), thereby forming a dehydration product comprising one or more C₄ alkenes and water;
(e) dimerizing, in the presence of an oligomerization catalyst, a dimerization feedstock comprising at least a portion of the C₄ alkenes formed in step (d), thereby forming a dimerization product comprising one or more C₈ alkenes;
(f) dehydrocyclizing, in the presence of a dehydrocyclization catalyst, a dehydrocyclization feedstock comprising at least a portion of the C₈ alkenes of step (e), thereby forming a dehydrocyclization product comprising xylenes and hydrogen, wherein the xylenes comprise at least about 75% p-xylene.

2. The method of claim 1, wherein the dimerization product of step (e) further comprises one or more unreacted C₄ alkenes, and the dehydrocyclization product further comprises one or more unreacted C₈ alkenes, and the method further comprises:
recycling at least a portion of the unreacted C₄ alkene(s) of the dimerization product and/or the unreacted C₈ alkene(s) of the dehydrocyclization product to the dimerization feedstock of step (e); and
(ii) recycling at least a portion of the unreacted C₈ alkene(s) of the dehydrocyclization product to the dehydrocyclization feedstock of step (f).

3. The method of claim 1, wherein at least about 95% of the one or more C₄ alkenes the dehydration product comprise isobutylene.

4. The method of claim 1, wherein said dehydrating of step (d) is carried out in the vapor phase, thereby producing isobutylene vapor and water.

5. The method of claim 1, wherein said dehydrating of step (d) is carried out in the liquid phase, thereby producing liquid isobutylene and water.

6. The method of claim 4, wherein after said dehydrating of step (d), at least a portion of the water produced thereby is removed from the isobutylene vapor using a gas-liquid separator.

7. The method of claim 5, wherein after said dehydrating step (d), a water rich phase is separated from an isobutylene rich phase using a liquid-liquid separator.

8. The method of claim 4, wherein the isobutylene vapor is condensed prior to said dimerizing of step (e).

9. The method of claim 4, wherein the isobutylene vapor and water are condensed after said dehydrating of step (d), prior to said dimerizing of step (e) a water rich phase is separated from an isobutylene rich phase using a liquid-liquid separator, and the dimerization feedstock comprises at least a portion of the isobutylene rich phase.

10. The method of claim 1, further comprising adding to the dimerization feedstock of step (e) at least one diluent selected from the group consisting of t-butanol, isobutanol, water, at least one hydrocarbon, and combinations thereof.

11. The method of claim 10, wherein the at least one diluent comprises at least one hydrocarbon, and the at least one hydrocarbon comprises at least one C₄ alkene recycled from the dimerization product of step (e) or the dehydrocyclization product of step (f), at least one C₄ alkane and/or C₈ alkane recycled from the dehydrocyclization product of step (f), or combinations thereof.

12. The method of claim 10, wherein the diluent comprises water and isobutanol.

13. The method of claim 2, further comprising adding to the dimerization feedstock of step (e) at least one diluent selected from the group consisting of t-butanol, isobutanol, water, at least one hydrocarbon, and combinations thereof.

14. The method of claim 13, wherein the at least one diluent comprises at least one hydrocarbon, and the at least one hydrocarbon comprises at least one C₄ alkene recycled from step (e) or step (f), at least one C₄ alkane and/or C₈ alkane recycled from step (f), or combinations thereof.

15. The method of claim 1, wherein the at least one or more C₈ alkenes of the dimerization product comprises about 50-100% of 2,4,4-trimethylpentenes.

16. The method of claim 15, wherein the at least one or more C₈ alkenes of the dimerization product comprises at least about 75% of 2,4,4-trimethylpentenes.

17. The method of claim 15, wherein the at least one or more C₈ alkenes of the dimerization product comprises at least about 90% of 2,4,4-trimethylpentenes.

18. The method of claim 1, wherein the at least one or more C₈ alkenes of the dimerization product comprises at least about 50-100% of 2,5-dimethylhexene.

19. The method of claim 18, wherein the at least one or more C₈ alkenes of the dimerization product comprises at least about 75% of 2,5-dimethylhexene.

20. The method of claim 18, wherein the at least one or more C₈ alkenes of the dimerization product comprises at least about 90% of 2,5-dimethylhexene.

21. The method of claim 1, wherein the at least one or more C₈ alkenes of the dimerization product comprises at least about 50-100% of 2,5-dimethylhexadiene.

22. The method of claim 21, wherein the at least one or more C₈ alkenes of the dimerization product comprises at least about 75% of 2,5-dimethylhexadiene.

23. The method of claim 21, wherein the at least one or more C₈ alkenes of the dimerization product comprises at least about 90% of 2,5-dimethylhexadiene.

24. The method of claim 1, further comprising adding to the dehydrocyclization feedstock of step (f) at least one diluent selected from the group consisting of nitrogen, argon, methane, isobutylene, isobutane, isooctane, light aromatics, and combinations thereof.

25. The method of claim 24, wherein the at least one diluent comprises isobutylene, which is unreacted isobutylene from steps (e) and/or (f), or a byproduct from step (f).

26. The method of claim 1, wherein:
said dehydrocyclization of step (f) is carried out at a conversion of less than about 100%; and
unreacted C₈ alkenes are recycled back to the dehydrocyclization feedstock of step (f).

27. The method of claim 1, wherein steps (e) and (f) are carried out simultaneously.

28. The method of claim 1, wherein steps (e) and (f) are carried out sequentially.

29. The method of claim 1, wherein the xylenes of the dehydrocyclization product comprise at least about 90% p-xylene.

30. The method of claim 1, wherein said dehydrating is carried out at temperature of at least about 100° C. and a pressure of at least about 1 atm.

31. The method of claim 1, wherein the dehydration catalyst is an organic or inorganic acid, or a metal salt thereof.

32. The method of claim 26, wherein the dehydration catalyst is a heterogeneous acidic γ-alumina catalyst.

33. The method of claim 1, wherein the oligomerization catalyst is a heterogeneous acidic catalyst.

34. The method of claim 33, wherein the oligomerization catalyst is an acidic zeolite, solid phosphoric acid, or a sulfonic acid resin.

35. The method of claim 1, wherein the dehydrocyclization catalyst is a heterogeneous metal-containing dehydrogenation catalyst.

36. The method of claim 35, wherein the dehydrocyclization catalyst is a supported chromium-containing compound.

37. The method of claim 33, wherein the dehydrocyclization catalyst is selected from the group consisting of chromium-oxide treated alumina; platinum- and tin-containing zeolites; and alumina, cobalt- or molybdenum-containing alumina.

38. The method of claim 1, wherein the aqueous isobutanol removed in step (c) consists essentially of isobutanol and 0-15% water.

39. The method of claim 1, further comprising hydrogenating an alkene in the presence of dehydrogenation catalyst with the hydrogen from step (f).

40. The method of claim 27, wherein said steps (e) and (f) are carried out simultaneously under oxidizing conditions.

41. The method of claim 40, wherein steps (e) and (f) are carried out in the presence of a single catalyst comprising bismuth oxide.

42. The method of claim 41, wherein the C₄ alkenes comprise isobutylene.

43. A method of preparing renewable terephthalic acid comprising:
preparing renewable p-xylene by the method of claim 1, then oxidizing the p-xylene in the presence of an oxidizing agent, thereby forming renewable terephthalic acid.

44. The method of claim 43, wherein the oxidizing agent comprises an oxidation catalyst and oxygen.

45. A method of preparing a renewable polyester comprising:
reacting renewable terephthalic acid prepared by the method of claim 40 with ethylene glycol or butylene glycol in the presence of an acidic polymerization catalyst.

46. The method of claim 45, wherein the acidic polymerization catalyst is antimony (III) oxide.

47. The method of claim 45, wherein the polyester is polyethylene terephthalate, and the ethylene glycol is renewable ethylene glycol.

48. The method of claim 45, wherein the polyester is polypropylene terephthalate, and the propylene glycol is renewable propylene glycol.

49. The method of claim 1, further comprising hydrogenating a portion of the dimerization product with at least a portion of the hydrogen of the dehydrocyclization product.
